(19) United States
(12) Patent Application Publication
(10) Pub. No.: US 2006/0063791 A1
(43) Pub. Date: Mar. 23, 2006

(54) (Spirocyclylamido)aminothiophene compounds

(76) Inventors: An-Hu Li, Farmingdale, NY (US); Hanqing Dong, Farmingdale, NY (US); Tao Zhang, Farmingdale, NY (US)

Correspondence Address: OSI PHARMACEUTICALS, INC., 58 SOUTH SERVICE ROAD, MELVILLE, NY, 11747, US

(21) Appl. No.: 11/227,345

(22) Filed: Sep. 15, 2005

Related U.S. Application Data
(60) Provisional application No. 60/610,672, filed on Sep. 17, 2004.

(51) Int. Cl. A61K 31/4747; C07D 409/14

(52) U.S. Cl. 514/278; 546/16

(57) ABSTRACT

Compounds represented by Formula (I): 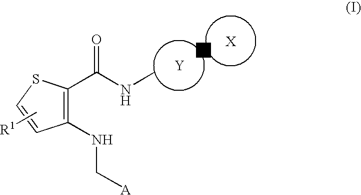
or a pharmaceutically acceptable salt or N-oxide thereof, wherein A, R¹, X and Y are defined herein, are useful in the treatment of tumors and cancers such as mastocytosis/mast cell leukemia, gastrointestinal stromal tumors (GIST), germ cell tumors, small cell lung carcinoma (SCLC), sinonasal natural killer/T-cell lymphoma, testicular cancer (seminoma), thyroid carcinoma, malignant melanoma, ovarian carcinoma, adenoid cystic carcinoma, acute myelogenous leukemia (AML), breast carcinoma, pediatric T-cell acute lymphoblastic leukemia, neuroblastoma, mast cell leukemia, angiosarcoma, anaplastic large cell lymphoma, endometrial carcinoma, and prostate carcinoma.

This application claims the benefit of U.S. Patent Application No. 60/610,672 filed 17 Sep. 2004.

### BACKGROUND OF THE INVENTION

The present invention is directed to disubstituted thiophenes. In particular, the present invention is directed to (spirocyclylamido)(amino)thiophenes that are inhibitors of c-Kit proto-oncogene (also known as Kit, CD-117, stem cell factor receptor, mast cell growth factor receptor).

The c-Kit proto-oncogene is believed to be important in embryogenesis, melanogenesis, hematopoiesis, and the pathogenesis of mastocytosis, gastrointestinal tumors, and other solid tumors, as well as certain leukemias, including AML. Accordingly, it would be desirable to develop novel compounds that are inhibitors of the c-Kit receptor.

Many of the current treatment regimes for hyperproliferative disorders (cancer) utilize compounds that inhibit DNA synthesis. Such compounds' mechanism of operation is to be toxic to cells, particularly to rapidly dividing tumor cells. Thus, their broad toxicity can be a problem to the subject patient. However, other approaches to anti-cancer agents that act other than by the inhibition of DNA synthesis have been explored to try to enhance the selectivity of the anti-cancer action and thereby reduce adverse side-effects.

It is known that a cell may become cancerous by virtue of the transformation of a portion of its DNA into an oncogene (i.e. a gene which, on activation, leads to the formation of malignant tumor cells). Many oncogenes encode proteins that are aberrant protein-tyrosine kinases capable of causing cell transformation. By a different route, the overexpression of a normal proto-oncogenic tyrosine kinase can also result in proliferative disorders, sometimes resulting in a malignant phenotype. Alternatively, co-expression of a receptor tyrosine kinase and its cognate ligand within the same cell type may also lead to malignant transformation.

Receptor tyrosine kinases are large enzymes which span the cell membrane and possess i) an extracellular binding domain for growth factors such as KIT ligand (also known as stem cell factor (SCF), Steel factor (SLF) or mast cell growth factor (MGF)), ii) a transmembrane domain, and iii) an intracellular portion which functions as a kinase to phosphorylate specific tyrosine residues in proteins. Binding of KIT ligand to KIT tyrosine kinase results in receptor homodimerization, the activation of KIT tyrosine kinase activity, and the subsequent phosphorylation of a variety of protein substrates, many of which are effectors of intracellular signal transduction, These events can lead to enhanced cell proliferation or promote enhanced cell survival. With some receptor kinases, receptor heterodimerization can also occur.

It is known that such kinases are frequently aberrantly expressed in common human cancers such as breast cancer, head and neck cancers, gastrointestinal cancer such as colon, rectal or stomach cancer, leukemia, and ovarian, bronchial, lung or pancreatic cancer. Kit kinase expression has been documented in a wide variety of human malignancies such as mastocytosis/mast cell leukemia, gastrointestinal stromal tumors (GIST), small cell lung carcinoma (SCLC), sinonasal natural killer/T-cell lymphoma, testicular cancer (seminoma), thyroid carcinoma, malignant melanoma, ovarian carcinoma, adenoid cystic carcinoma, acute myelogenous leukemia (AML), breast carcinoma, pediatric T-cell acute lymphoblastic leukemia, angiosarcoma, anaplastic large cell lymphoma, endometrial carcinoma, and prostate carcinoma. The kinase activity of KIT has been implicated in the pathophysiology of several of these—and additional tumors—including breast carcinoma, SCLC, GIST, germ cell tumors, mast cell leukemia, neuroblastoma, AML, melanoma and ovarian carcinoma.

Several mechanisms of KIT activation in tumor cells have been reported, including activating mutations, autocrine and paracrine activation of the receptor kinase by its ligand, loss of protein-tyrosine phosphatase activity, and cross activation by other kinases. The transforming mechanisms initiated by the activating mutations are thought to include dimer formation and increased intrinsic activity of the kinase domain, both of which result in constitutive ligand-independent kinase activation, and possibly altered substrate specificity. More than thirty activating mutations of the Kit protein have been associated with highly malignant tumors in humans.

Accordingly, it has been recognized that inhibitors of receptor tyrosine kinases are useful as selective inhibitors of the growth of mammalian cancer cells. For example, Gleevec™ (also known as imatinib mesylate, or STI571), a 2-phenylpyrimidine tyrosine kinase inhibitor that inhibits the kinase activity of the BCR-ABL fusion gene product, was recently approved by the U.S. Food and Drug Administration for the treatment of CML. Gleevec™, in addition to inhibiting BCR-ABL kinase, also inhibits the KIT kinase and PDGF receptor kinase, although it is not effective against all mutant isoforms of the KIT kinase. Kit ligand-stimulated growth of MO7e human leukemia cells is inhibited by Gleevec™, which also induces apoptosis under these conditions. By contrast, GM-CSF stimulated growth of MO7e human leukemia cells is not affected by Gleevec™. Further, in recent clinical studies using Gleevec™ to treat patients with GIST, a disease in which KIT kinase is involved in transformation of the cells, many of the patients showed marked improvement.

These studies demonstrate how KIT kinase inhibitors can treat tumors whose growth is dependent on KIT kinase activity. Other kinase inhibitors show even greater kinase selectivity. For example, the 4-anilinoquinazoline compound Tarceva™ inhibits only EGF receptor kinase with high potency, although it can inhibit the signal transduction of other receptor kinases, probably by virtue of the fact that these receptors heterodimerize with EGF receptor.

Although anti-cancer compounds such as those described above make a significant contribution to the art, there is a continuing need for improved anti-cancer pharmaceuticals, and it would be desirable to develop new compounds with better selectivity or potency, or with reduced toxicity or side effects.

International Patent Publication No. WO02/00651 describes factor Xa inhibitors. U.S. Pat. No. 6,054,457 describes benzamide derivatives and their use as vasopressin antagonists.

### SUMMARY OF THE INVENTION

Compounds represented by Formula (I): 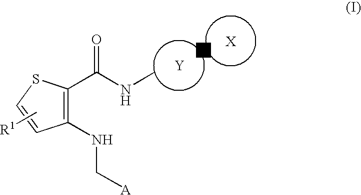
or a pharmaceutically acceptable salt or N-oxide thereof. The compounds of Formula (I) are useful in the treatment of tumors and cancers such as mastocytosis/mast cell leukemia, gastrointestinal stromal tumors (GIST), germ cell tumors, small cell lung carcinoma (SCLC), sinonasal natural killer/T-cell lymphoma, testicular cancer (seminoma), thyroid carcinoma, malignant melanoma, ovarian carcinoma, adenoid cystic carcinoma, acute myelogenous leukemia (AML), breast carcinoma, pediatric T-cell acute lymphoblastic leukemia, neuroblastoma, mast cell leukemia, angiosarcoma, anaplastic large cell lymphoma, endometrial carcinoma, and prostate carcinoma.

### DETAILED DESCRIPTION OF THE INVENTION

The present invention is directed to a compound represented by Formula (I): 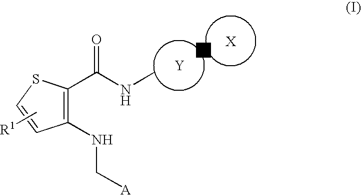
or a pharmaceutically acceptable salt or N-oxide thereof, wherein:
Y is heteroaryl or cycloC₃₋₁₀alkyl, either of which is optionally substituted by 1-5 independent R² substituents;
X is heteroaryl or heterocylyl, either of which is optionally substituted by 1-5 independent R²¹ substituents;
A is aryl, heteroaryl, cycloC₃₋₁₀alkyl, heterocyclyl, cycloC₃₋₁₀alkenyl, or heterocycloalkenyl, each of which is optionally substituted by 1-5 independent R³ substituents;
R¹ is C₀₋₆alkyl, halogen, or haloalkyl;
R², R²¹, and R³ each independently is C₀₋₆alkyl, cycloC₃₋₁₀alkyl, oxo, halogen, haloalkyl, cyanoC₀₋₆alkyl, nitroC₀₋₆alkyl, hydroxyC₀₋₆alkyl, —C₀₋₆alkyl-N(C₀₋₆alkyl)(C₀₋₆alkyl), —N(C₀₋₆alkyl)-N(C₀₋₆alkyl)(C₀₋₆alkyl), —N(C₀₋₆alkyl)-N(C₀₋₆alkyl)(acyl), acylC₀₋₆alkyl, substituted acyl, guanidinoC₀₋₆alkyl, hydroxyiminoC₀₋₆alkyl, acylaminoC₀₋₆alkyl, substituted acylamino, acyloxyC₀₋₆alkyl, substituted acyloxy, arC₀₋₆alkyl, substituted arC₀₋₆alkyl, heteroarylC₀₋₆alkyl, substituted heteroarylC₀₋₆alkyl, heterocyclylC₀₋₆alkyl, cyanoaminoC₀₋₆alkyl, C₀₋₆alkylhydrazino, heterocyclylamino, arC₀₋₆alkylhydrazino, alkylsulfonylC₀₋₆alkyl, arC₀₋₆alkylsulfonylC₀₋₆alkyl, alkylsulfinylC₀₋₆alkyl, alkylsulfonamidoC₀₋₆alkyl, arC₀₋₆alkylsulfonanmidoC₀₋₆alkyl, aminoC₀₋₆alkylsulfonyl, C₀₋₆alkylaminosulfonyl, acylC₁₋₆alkylsulfonyl, heterocyclylsulfonyl, aminoC₀₋₆alkylsulfinyl, acylC₁₋₆alkylsulfinyl, silyl, siloxy, alkenoxy, alkynoxy, C₂₋₆alkenyl, acylC₂₋₆alkenyl, C₂₋₆alkynyl, acylC₂₋₆alkynyl, hydroxyC₂₋₆alkynyl, aminoC₂₋₆alkynyl, C₁₋₆alkoxyC₀₋₆alkyl, C₁₋₆alkylthioC₀₋₆alkyl, hydroxyC₁₋₆alkoxyC₀₋₆alkyl, hydroxyC₁₋₆alkylthioC₀₋₆alkyl, acylC₁₋₆alkoxyC₀₋₆alkyl, acylC₁₋₄alkylthioC₀₋₆alkyl, C₀₋₆alkylaminoC₁₋₆alkoxyC₀₋₆alkyl, C₀₋₆alkylaminoC₁₋₆alkylthioC₀₋₆alkyl, acylaminoC₁₋₆alkoxyC₀₋₆alkyl, acylaminoC₁₋₆alkylthioC₀₋₆alkyl, arC₀₋₆alkylaminoC₀₋₆alkyl, arC₀₋₆alkylthioC₀₋₆alkyl, arC₀₋₆alkoxyC₀₋₆alkyl, arC₀₋₆alkylamino, arC₀₋₆alkylaminoC₀₋₆alkyl, arC₀₋₆alkylthio, substituted arC₀₋₆alkoxy, substituted arC₀₋₆alkylthio, or substituted arC₀₋₆alkoxy;

provided that the compound is not
cis-N-(4-methyl-5-oxo-1,4-diazaspiro[5.5]undec-9-yl)-3-[(quinolin-4-ylmethyl)amino]thiophene-2-carboxamide,
cis-N-(2-benzyl-3-oxo-1,2-diazaspiro[4.5]dec-8-yl)-3-[(quinolin-4-ylmethyl)amino]thiophene-2-carboxamide,
N-(3-benzyl-2,4-dioxo-1,3-diazaspiro[4.5]dec-8-yl)-3-[(quinolin-4-ylmethyl)amino]thiophene-2-carboxamide,
cis-N-(2,4-dioxo-1,3-diazaspiro[4.5]dec-8-yl)-3-[(quinolin-4-ylmethyl)amino]thiophene-2-carboxamide,
N-[3-(3-methylbutyl)-2,4-dioxo-1,3-diazaspiro[4.5]dec-8-yl]4-methyl-3-[(quinolin-4-ylmethyl)amino]thiophene-2-carboxamide, or
benzyl 5-{[(3-[(quinolin-4-ylmethyl)amino]thien-2-yl)carbonyl]amino}-2-oxo-1,2-dihydro-1′H-spiro[indole-3,4′-piperidine]-1′-carboxylate.

In one aspect, the present invention is directed to a compound represented by Formula (I), or a pharmaceutically acceptable salt or N-oxide thereof, wherein Y is cycloC₃₋₁₀alkyl optionally substituted by 1-5 independent R² substituents; X is heterocyclyl or heteroaryl optionally substituted by 1-5 independent R²¹ substituents; and the other variables are as described above for Formula (I).

In an embodiment of this one aspect, the present invention is directed to a compound represented by Formula (I), or a pharmaceutically acceptable salt or N-oxide thereof, wherein Y is cyclohexyl optionally substituted by 1-5 independent R² substituents; X is heterocyclyl or heteroaryl; and the other variables are as described above for Formula (I).

In another embodiment of this one aspect, the present invention is directed to a compound represented by Formula (I), or a pharmaceutically acceptable salt or N-oxide thereof, wherein Y is cyclohexyl optionally substituted by 1-5 independent R² substituents; X is heterocyclyl or heteroaryl optionally substituted by 1-5 independent R²¹ substituents; A is heteroaryl optionally substituted by 1-5 independent R³ substituents; and the other variables are as described above for Formula (I).

In still another embodiment of this one aspect, the present invention is directed to a compound represented by Formula (I), or a pharmaceutically acceptable salt or N-oxide thereof, wherein Y is cyclohexyl optionally substituted by 1-5 independent R² substituents; X is heterocyclyl or heteroaryl optionally substituted by 1-5 independent R²¹ substituents; A is 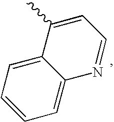
and the other variables are as described above for Formula (I).

In yet another embodiment of this one aspect, the present invention is directed to a compound represented by Formula (I), or a pharmaceutically acceptable salt or N-oxide thereof, wherein Y is cyclohexyl optionally substituted by 1-5 independent R² substituents; X is heterocyclyl or heteroaryl; R¹ is hydrogen; A is 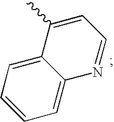
and the other variables are as described above for Formula (I).

In another embodiment of this one aspect, the present invention is directed to a compound represented by Formula (I), or a pharmaceutically acceptable salt or N-oxide thereof, wherein Y is cyclohexyl optionally substituted by 1-5 independent R² substituents; X is heterocyclyl or heteroaryl optionally substituted by 1-5 independent R²¹ substituents; R¹ is C₁₋₆alkyl; and the other variables are as described above for Formula (I).

In yet still another embodiment of this one aspect, the present invention is directed to a compound represented by Formula (I), or a pharmaceutically acceptable salt or N-oxide thereof, wherein Y is cyclohexyl optionally substituted by 1-5 independent R² substituents; X is heterocyclyl optionally substituted by 1-5 independent R²¹ substituents; R¹ is C₀₋₆alkyl; and the other variables are as described above for Formula (I).

In another embodiment of this one aspect, the present invention is directed to a compound represented by Formula (I), or a pharmaceutically acceptable salt or N-oxide thereof, wherein Y is cyclohexyl optionally substituted by 1-5 independent R² substituents; X is heteroaryl optionally substituted by 1-5 independent R² substituents; RX is C₀₋₆alkyl; and the other variables are as described above for Formula (I).

In a second aspect, the present invention is directed to a compound represented by Formula (I), or a pharmaceutically acceptable salt or N-oxide thereof, wherein Y is heteroaryl optionally substituted by 1-5 independent R² substituents; X is heterocyclyl optionally substituted by 1-5 independent R²¹ substituents; and the other variables are as described above for Formula (I).

In an embodiment of this second aspect, the present invention is directed to a compound represented by Formula (I), or a pharmaceutically acceptable salt or N-oxide thereof, wherein Y is heteroaryl optionally substituted by 1-5 independent R² substituents; X is heterocyclyl optionally substituted by 1-5 independent R²¹ substituents; R¹ is C₀₋₆alkyl; A is heteroaryl optionally substituted by 1-5 independent R³ substituents; and the other variables are as described above for Formula (I).

In an embodiment of this second aspect, the present invention is directed to a compound represented by Formula (I), or a pharmaceutically acceptable salt or N-oxide thereof, wherein Y is heteroaryl optionally substituted by 1-5 independent R² substituents; X is heterocyclyl optionally substituted by 1-5 independent R²¹ substituents; A is 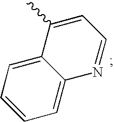
and the other variables are as described above for Formula (I).

In an embodiment of this second aspect, the present invention is directed to a compound represented by Formula (I), or a pharmaceutically acceptable salt or N-oxide thereof, wherein Y is heteroaryl optionally substituted by 1-5 independent R² substituents; X is heterocyclyl optionally substituted by 1-5 independent R²¹ substituents; R¹ is hydrogen; A is 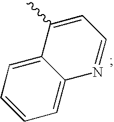
and the other variables are as described above for Formula (I).

The present invention includes the following compounds:
N-1,4-dioxaspiro[4.5]dec-8-yl-3-[(quinolin-4-ylmethyl)amino]thiophene-2-carboxamide;
cis-N-1-oxa-4-thiaspiro[4.5]dec-8-yl-3-[(quinolin-4-ylmethyl)amino]thiophene-2-carboxamide;
trans-N-1-oxa-4-thiaspiro[4.5]dec-8-yl-3-[(quinolin-4-ylmethyl)amino]thiophene-2-carboxamide;
N-(2-methyl-3-oxo-1,2-diazaspiro[4.5]dec-8-yl)-3-[(quinolin-4-ylmethyl)amino]thiophene-2-carboxamide;
trans-N-(2-benzyl-3-oxo-1,2-diazaspiro[4.5]dec-8-yl)-3-[(quinolin-4-ylmethyl)amino]thiophene-2-carboxamide;
cis-N-(1,4-dimethyl-5-oxo-1,4-diazaspiro[5.5]undec-9-yl)-3-[(quinolin-4-ylmethyl)amino]thiophene-2-carboxamide;
trans-N-(1,4-dimethyl-5-oxo-1,4-diazaspiro[5.5]undec-9-yl)-3-[(quinolin-4-ylmethyl)amino]thiophene-2-carboxamide;
cis-N-(3′-oxo-3′,4′-dihydro-1′H-spiro[cyclohexane-1,2′-quinoxalin]4-yl)-3-[(quinolin-4-ylmethyl)amino]thiophene-2-carboxamide;
trans-N-(3′-oxo-3′,4′-dihydro-1′H-spiro[cyclohexane-1,2′-quinoxalin]4-yl)-3-[(quinolin-4-ylmethyl)amino]thiophene-2-carboxamide;
N-(3-oxo-2-azaspiro[4.5]dec-8-yl)-3-[(quinolin-4-ylmethyl)amino]thiophene-2-carboxamide;
N−1,4-dioxaspiro[4.5]dec-8-yl-4-methyl-3-[(quinolin-4-ylmethyl)amino]thiophene-2-carboxamide;
trans-N-(2,4-dioxo-1,3-diazaspiro[4.5]dec-8-yl)-3-[(quinolin-4-ylmethyl)amino]thiophene-2-carboxamide;
N-(3-methyl-2,4-dioxo-1,3-diazaspiro[4.5]dec-8-yl)-3-[(quinolin-4-ylmethyl)amino]thiophene-2-carboxamide;
N-[3-(3-methylbutyl)-2,4-dioxo-1,3-diazaspiro[4.5]dec-8-yl]-3-[(quinolin-4-ylmethyl)amino]thiophene-2-carboxamide;
N-(1′-methyl-2-oxo-1,2-dihydrospiro[indole-3,4′-piperidin]-5-yl)-3-[(quinolin-4-ylmethyl)amino]thiophene-2-carboxamide;
N-(2′-oxo-1′,2′-dihydrospiro[1,3-dioxolane-2,3′-indol]-5′-yl)-3-[(quinolin-4-ylmethyl)amino]thiophene-2-carboxamide;
N-1′,2′-dihydrospiro[1,3-dioxolane-2,3′-indol]-5′-yl-3-[(pyridin-4-ylmethyl)amino]thiophene-2-carboxamide;
N-(2-oxo-1,2,2′,3′,5′,6′-hexahydrospiro[indole-3,4′-pyran]-5-yl)-3-[(quinolin-4-ylmethyl)amino]thiophene-2-carboxamide;
N-(2′-oxo-1′,2′-dihydrospiro[1,3-dithiolane-2,3′-indol]-5′-yl)-3-[(quinolin-4-ylmethyl)amino]thiophene-2-carboxamide;
N-(1,3-dimethyl-2′-oxo-1′,2′-dihydrospiro[imidazolidine-2,3′-indol]-5′-yl)-3-[(quinolin-4-ylmethyl)amino]thiophene-2-carboxamide;
3-[(quinolin-4-ylmethyl)amino]-N-(1,1′,3-trimethyl-2′-oxo-1′,2′-dihydrospiro[imidazolidine-2,3′-indol]-5′-yl)thiophene-2-carboxamide;
N-(2′-oxo-1′,2′-dihydrospiro[1,3-dioxane-2,3′-indol]-5′-yl)-3-[(quinolin-4-ylmethyl)amino]thiophene-2-carboxamide;
4-methyl-N-(2′-oxo-1′,2′-dihydrospiro[1,3-dioxolane-2,3′-indol]-5′-yl)-3-[(quinolin-4-ylmethyl)amino]thiophene-2-carboxamide;
4-methyl-N-(2′-oxo-1′,2′-dihydrospiro[1,3-dithiolane-2,3′-indol]-5′-yl)-3-[(quinolin-4-ylmethyl)amino]thiophene-2-carboxamide;
N-(1,3-dimethyl-2′-oxo-1′,2′-dihydrospiro[imidazolidine-2,3′-indol]-5′-yl)-4-methyl-3-[(quinolin-4-ylmethyl)amino]thiophene-2-carboxamide;
4-methyl-3-[(quinolin-4-ylmethyl)amino]-N-(1,1′,3-trimethyl-2′-oxo-1′,2′-dihydrospiro[imidazolidine-2,3′-indol]-5′-yl)thiophene-2-carboxamide
   or a pharmaceutically acceptable salt, or N-oxide, thereof.

The present invention is also directed to a method of treating hyperproliferative disorders, including breast cancer, head cancer, or neck cancer, gastrointestinal cancer, leukemia, ovarian, bronchial, lung, or pancreatic cancer, mastocytosis/mast cell leukemia, gastrointestinal stromal tumors (GIST), germ cell tumors, small cell lung carcinoma (SCLC), sinonasal natural killer/f-cell lymphoma, testicular cancer (seminoma), thyroid carcinoma, malignant melanoma, ovarian carcinoma, adenoid cystic carcinoma, acute myelogenous leukemia (AML), breast carcinoma, pediatric T-cell acute lymphoblastic leukemia, neuroblastoma, mast cell leukemia, angiosarcoma, anaplastic large cell lymphoma, endometrial carcinoma, and prostate carcinoma, by administering an effective amount of a compound represented by Formula (I), or a pharmaceutically acceptable salt thereof.

As used herein, “C₀₋₆alkyl” is used to mean an alkyl having 0-6 carbons—that is, 0, 1, 2, 3, 4, 5, or 6 carbons in a straight or branched configuration. An alkyl having no carbon is hydrogen when the alkyl is a terminal group. An alkyl having no carbon is a direct bond when the alkyl is a bridging (connecting) group.

As used herein unless otherwise specified, “alkyl”, “alkenyl”, and “alkynyl” includes straight or branched configurations. Lower alkyls, alkenyls, and alkynyls have 1-6 carbons. Higher alkyls, alkenyls, and alkynyls have more than 6 carbons.

As used herein unless otherwise specified, “halogen” is fluorine, chlorine, bromine or iodine.

As used herein unless otherwise specified, “substituted” is used to mean having 1-5 independent C₀₋₆alkyl, halogen, nitro, cyano, haloalkyl, C₀₋₆alkoxy, C₀₋₆alkylthio, or C₀₋₆alkylamino substituents

As used herein unless otherwise specified, “haloalkyl” includes alkyl groups substituted with one or more halogens, for example, chloromethyl, 2-bromoethyl, 3-iodopropyl, trifluoromethyl, perfluoropropyl, 8-chlorononyl, and the like.

As used herein unless otherwise specified, the terms “aryl” and “ar” are well known to chemists and include, for example, phenyl and naphthyl, as well as phenyl with one or more short alkyl groups (tolyl, xylyl, mesityl, cumenyl, di(t-butyl)phenyl). Phenyl, naphthyl, tolyl, and xylyl are preferred. “Substituted aryl” is an aryl substituted with suitable substituents such as, for example, acyl, substituted acyl, N-protected piperazinylsulfonyl, piperazinylsulfonyl, N—C₁₋₆alkylpiperazinylsulfonyl, hydroxyC₁₋₆alkyl, heterocyclyl, halogen, nitro, amino, C₁₋₆alkylamino, cyano, or C₁₋₆alkoxy.

As used herein unless otherwise specified, the term “cycloalkyl” is well known to chemists and includes cyclic aliphatic ring structures, optionally substituted with alkyl, hydroxyl, oxo, and halo, such as cyclopropyl, methylcyclopropyl, cyclobutyl, cyclopentyl, 2-hydroxycyclopentyl, cyclopentanonyl, cyclohexyl, 4-chlorocyclohexyl, cycloheptyl, cyclooctyl, and the like.

As used herein unless otherwise specified, the term “cycloalkyl” is well known to chemists and includes cyclic aliphatic ring structures having at least one ethylenic bond, optionally substituted with alkyl, hydroxyl, oxo, and halo, for example, methylcyclopropenyl, trifluoromethylcyclopropenyl, cyclopentenyl, cyclohexenonyl, cyclohexenyl, 1,4-cyclohexadienyl, and the like.

As used herein unless otherwise specified, “heterocyclyl” is well known to chemists and includes unsaturated, mono or polycyclic heterocyclic groups containing at least one N, S or O hetero-ring atom such as, for example, tetrahydrofuranyl, tetrahydrofuryl, pyrrolidinyl, piperidinyl, tetrahydropyranyl, thiolanyl, morpholinyl, piperazinyl, homopiperazinyl, dioxolanyl, dioxanyl, indolinyl, or chromanyl and the like. Such heterocyclyls can be suitably substituted with lower alkyl or oxo substituents.

As used herein unless otherwise specified, “heteroaryl” is well known to chemists and includes partially saturated, mono or polycyclic heterocyclic groups containing at least one N, S or O hetero-ring atom such as, for example, pyrazolyl, pyridyl, pyrimidinyl, pyrazinyl, pyridazinyl, triazolyl, tetrazolyl, pyrrolidinyl, indolyl, indolinyl, isoindolyl, indolizinyl, benzimidazolyl, quinolyl, isoquinolyl, imidazopyridyl, indazolyl, benzotriazolyl, tetrazolo-pyridazinyl, pyranyl, furyl, thienyl, oxazolyl, isoxazolyl, oxazolyl, benzofuranyl, benzoxazolyl, benzoxadiazolyl, thiazolyl, thiadiazolyl, thiazolidinyl, benzothiazolyl, benzothiadiazolyl, benzofuranyl, or benzodioxyl, imidazolyl, pyrrolyl, oxadiazolyl, quinolyl, benzotriazolyl, or benzothienyl and the like. Such heterocyclyls can be suitably substituted with lower alkyl or oxo substituents.

As used herein unless otherwise specified, “heterocycloalkenyl” includes mono or polycyclic heterocyclic groups having at least one ethylenic bond and containing at least one N, S or O hetero-ring atom such as, for example, dihydropyranyl, dihydrofuran, pyrrolinyl or the like. Such heterocycloalkenyls can be suitably substituted with lower alkyl or oxo substituents.

As used herein unless otherwise specified, “acyl” includes for example, carboxy, esterified carboxy, carbamoyl, lower alkylcarbamoyl, lower alkanoyl, aroyl, heterocyclylcarbonyl, and the like. Esterified carboxy includes substituted or unsubstituted lower alkoxycarbonyl such as methoxycarbonyl, ethoxycarbonyl, propoxycarbonyl, butoxycarbonyl, t-butoxycarbonyl, hexyloxycarbonyl, 2-iodoethoxycarbonyl, 2,2,2-trichloroethoxycarbonyl, dimethylaminopropoxycarbonyl, dimethylaminoethoxycarbonyl; substituted or unsubstituted aryloxycarbonyl such as phenoxycarbonyl, 4-nitrophenoxycarbonyl, 2-naphthyloxycarbonyl; substituted or unsubstituted ar(lower)alkoxycarbonyl such as benzyloxycarbonyl, phenethyloxycarbonyl, benzhydryloxycarbonyl, 4-nitrobenzyloxycarbonyl, 3-methoxy-4-nitrobenzyloxycarbonyl; and N-containing heterocyclyloxycarbonyl such as N-methylpiperidyloxycarbonyl and the like.

As used herein unless otherwise specified, “C₀₋₆alkylhydrazino” may be 2-mono or 2,2-di(C₀₋₆alkyl)hydrazino such as 2-methylhydrazino, 2,2-dimethylhydrazino, 2-ethylhydrazino, hydrazine, 2,2-diethylhydrazino, or the like.

As used herein unless otherwise specified, alkylamino such as “C₁₋₆alkylamino” may be mono or dialkylamino such as methylamino, dimethylamino, N-methylethylamino or the like. Similarly, other amino groups such as acylamino are understood to include a C₀₋₆alkyl at the unspecified amino bond site (one being to the acyl, the second forming a connection to the core structure, and the third unspecified).

As used herein unless otherwise specified, “arC₀₋₆alkylamino” may be mono or disubstitutedamino such as anilino, benzylamino, N-methylanilino, N-benzylmethylamino or the like.

As used herein unless otherwise specified, “silyl” includes alkyl and aryl substituted silyl groups such as, for example, triethylsilyl, t-butyldiphenylsilyl, or the like.

As used herein unless otherwise specified, “siloxy” includes alkyl and aryl substituted silyloxy groups such as, for example, triethylsilyloxy, t-butyldiphenylsilyloxy, or the like.

As used herein unless otherwise specified, “sulfonyloxy” includes sulfonyloxy groups substituted with aryl, substituted aryl, or alkyl such as, for example, benzenesulfonyl, tosyl, mesyl or the like.

As used herein unless otherwise specified, “heterocyclylamino” includes unsaturated, mono or polycyclic heterocyclic groups containing at least one N-ring atom which is attached to an amino group such as, for example, 1-aminopiperidine, 1-aminomorpholine, 1-amino-4-methylpiperazine or the like.

As used herein unless otherwise specified (for example, by a dash marking the point of attachment), chemical group names comprised of multiple chemical terms are used according to standard chemical convention, wherein each term modifies the following term and wherein the rightmost term forms a covalent bond with the structure to which the substituent is attached. For example, aralkylamino includes benzylamino and phenethylamino attached through the amino nitrogen, but not toluidino or N-methylanilino groups.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, methanesulfonic, and tartaric acids.

The pharmaceutical compositions of the present invention or used by the methods of the present invention comprise a compound represented by Formula (I) (or a pharmaceutically acceptable salt or N-oxide thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula (I), or pharmaceutically acceptable salts or N-oxides thereof, of this invention can be combined as the active ingredient in intimate admixture with a pharmaceutical carrier according to conventional pharmaceutical compounding techniques. The carrier may take a wide variety of forms depending on the form of preparation desired for administration. E.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula (I), or a pharmaceutically acceptable salt or N-oxide thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt or N-oxide of Formula (I). The compounds of Formula (I), or pharmaceutically acceptable salts or N-oxides thereof, can also be included in pharmaceutical compositions in combination with one or more other therapeutically active compounds.

The pharmaceutical compositions of this invention include a pharmaceutically acceptable liposomal formulation containing a compound of Formula (I) or a pharmaceutically acceptable salt or N-oxide thereof.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent or other such excipient. These excipients may be, for example, inert diluents such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example, magnesium stearate, stearic acid or talc. The tablets may be uncoated or they may be coated by known techniques to delay disintegration and absorption in the gastrointestinal tract and thereby provide a sustained action over a longer time. For example, a time delay material such as glyceryl monostearate or glyceryl distearate may be used.

In hard gelatin capsules, the active ingredient is mixed with an inert solid diluent, for example, calcium carbonate, calcium phosphate or kaolin. In soft gelatin capsules, the active ingredient is mixed with water or an oil medium, for example, peanut oil, liquid paraffin or olive oil. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material, which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula (I) of this invention, or a pharmaceutically acceptable salt or N-oxide thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula (I), or pharmaceutically acceptable salts or N-oxides thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 10 g per patient per day. For example, breast cancer, head and neck cancers, and gastrointestinal cancer such as colon, rectal or stomach cancer may be effectively treated by the administration of from about 0.01 to 100 mg of the compound per kilogram of body weight per day, or alternatively about 0.5 mg to about 7 g per patient per day.

Similarly, leukemia, ovarian, bronchial, lung, and pancreatic cancer may be effectively treated by the administration of from about 0.01 to 100 mg of the compound per kilogram of body weight per day, or alternatively about 0.5 mg to about 7 g per patient per day.

Mastocytosis/mast cell leukemia, gastrointestinal stromal tumors (GIST), small cell lung carcinoma (SCLC), sinonasal natural killer/T-cell lymphoma, testicular cancer (seminoma), thyroid carcinoma, malignant melanoma, ovarian carcinoma, adenoid cystic carcinoma, acute myelogenous leukemia (AML), breast carcinoma, pediatric T-cell acute lymphoblastic leukemia, angiosarcoma, anaplastic large cell lymphoma, endometrial carcinoma, and prostate carcinoma may be effectively treated by the administration of from about 0.01 to 100 mg of the compound per kilogram of body weight per day, or alternatively about 0.5 mg to about 7 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of the present invention, or pharmaceutically acceptable salts or N-oxides thereof, can also be effectively administered in conjunction with other cancer therapeutic compounds. For example, cytotoxic agents and angiogenesis inhibiting agents can be advantageous co-agents with the compounds of the present invention. Accordingly, the present invention includes compositions comprising the compounds represented by Formula (I), or a pharmaceutically acceptable salt or N-oxide thereof, and a cytotoxic agent or an angiogenesis-inhibiting agent. The amounts of each can be therapeutically effective alone—in which case the additive effects can overcome cancers resistant to treatment by monotherapy. The amounts of any can also be subtherapeutic—to minimize adverse effects, particularly in sensitive patients.

It is understood that the treatment of cancer depends on the type of cancer. For example, lung cancer is treated differently as a first line therapy than are colon cancer or breast cancer treated. Even within lung cancer, for example, first line therapy is different from second line therapy, which in turn is different from third line therapy. Newly diagnosed patients might be treated with cisplatinum containing regimens. Were that to fail, they move onto a second line therapy such as a taxane. Finally, if that failed, they might get a tyrosine kinase EGFR inhibitor as a third line therapy. Further, The regulatory approval process differs from country to country. Accordingly, the accepted treatment regimens can differ from country to country. Nevertheless, the compounds of the present invention, or pharmaceutically acceptable salts or N-oxides thereof, can be beneficially co-administered in conjunction or combination with other such cancer therapeutic compounds. Such other compounds include, for example, a variety of cytotoxic agents (alkylators, DNA topoisomerase inhibitors, antimetabolites, tubulin binders); inhibitors of angiogenesis; and different other forms of therapies including kinase inhibitors such as Tarceva, monoclonal antibodies, and cancer vaccines. Other such compounds that can be beneficially co-administered with the compounds of the present invention include doxorubicin, vincristine, cisplatin, carboplatin, gemcitabine, and the taxanes. Thus, the compositions of the present invention include a compound according to Formula (I), or a pharmaceutically acceptable salt or N-oxide thereof, and an anti-neoplastic, anti-tumor, anti-angiogenic, or chemotherapeutic agent.

The compounds of the present invention, or pharmaceutically acceptable salts or N-oxides thereof, can also be effectively administered in conjunction with other therapeutic compounds, aside from cancer therapy. For example, therapeutic agents effective to ameliorate adverse side-effects can be advantageous co-agents with the compounds of the present invention.

C-KIT H526 Cell Assay Protocol

I. Assay for Inhibition of c-Kit in Intact Cells

The ability of compounds to inhibit the tyrosine kinase activity of c-Kit was determined in a cell-based ELISA assay using the H526 cell line (ATCC # CRL-5811), which was originally derived from a human small cell lung cancer. The assay determines the ability of compounds to block ligand-stimulated tyrosine phosphorylation of the wild-type c-Kit receptor protein that is endogenously expressed in H526 cells. Cells are pre-incubated with compounds at various concentrations prior to addition of stem cell factor (SCF), the ligand for the c-Kit receptor tyrosine kinase. Cell lysates are then prepared and the c-Kit protein is captured onto a c-Kit antibody-coated 96-well ELISA plate. The phosphotyrosine content of the receptor protein is then monitored by quantitation of the degree of binding of an antibody that recognizes only the phosphorylated tyrosine residues within the captured protein. The antibody used has a reporter enzyme (e.g. horseradish peroxidase, HRP) covalently attached, such that binding of antibody to phosphorylated c-Kit can be determined quantitatively by incubation with an appropriate HRP substrate.

The stock reagents used are as follows:

Cell Lysis Buffer:
50 mM Tris-HCl, pH 7.4
150 mM NaCl
10% Glycerol
1% Triton X-100
0.5 mM EDTA
1 μg/mL leupeptin
1 μg/mL aprotinin
1 mM Sodium orthovanadate

Anti c-Kit Antibody:
0.5 μg/mL anti c-Kit Ab-3 (Lab Vision, catalog #MS289P1) in 50 mM Sodium bicarbonate, pH 9.

ELISA Assay Plates:
ELISA assay plates are prepared by addition of 100 μL of anti c-Kit antibody to each well of a 96-well Microlite-2 plate (Dynex, catalog # 7417), followed by incubation at 37° C. for 2 h. The wells are then washed twice with 300 μL wash buffer.

Plate Wash Buffer:
PBS containing 0.5% Tween-20 (PBST)

Cell Assay Medium:
RPMI with 0.1% BSA

pY20-HRP:
25 ng/mL pY20-HRP (Calbiochem, catalog # 525320) in PBS, containing 0.5% Tween-20, 5% BSA, 1 mM Sodium orthovanadate

HRP Substrate:
Chemoluminescent detection reagent (Pierce, catalog # 37075)

Assay Protocol

Cultures of H526 cells, growing in RPMI with 10% fetal calf serum, were collected by centrifugation, washed twice with PBS, and suspended in cell assay medium. Cells were then distributed into a V-bottom 96-well plate at 7.5×10⁴ cells per well in 100 μL cell assay medium.

Compound dilutions were prepared from 10 mM DMSO stocks by dilution in cell assay medium, the final concentration of DMSO in the assay being 0.1%. To compound incubation wells, 50 μL of the test compound was added (compounds are assayed at concentrations between 0.1 nM and 100 μM); to positive and negative control wells, 50 μL cell assay medium containing 0.1% DMSO was added. The cells were then incubated with compound at 37° C. for 3 h. SCF (R&D Systems, catalog #255-SC-010) was then added in order to stimulate the c-Kit receptor and induce its tyrosine phosphorylation. Then, 10 μL of a 1.6 μg/mL solution of SCF in cell assay medium was added to all wells apart from the negative control wells, and the cells were incubated for an additional 15 min at 37° C. Following the addition of ice-cold PBS, the plate was centrifuged at 1000 rpm for 5 min, the medium removed by aspiration, and the cell pellet lysed by the addition of 120 μL ice-cold cell lysis buffer per well. The plate was kept on ice for 20 min and 100 μL of the cell lysates from each well were then transferred to the wells of an ELISA assay plate and incubated at 4° C. for 16 h.

Following incubation of the cell lysates in the ELISA plate, the wells were washed 4 times with 300 μL wash buffer, then 100 μL of the phosphotyrosine detection antibody pY20-HRP was added to each well and the plate incubated at rt for 2 h. The wells were then washed 4 times with 300 μL wash buffer. Then, 50 μL of the chemiluminescent HRP substrate was added to each well for luminometric quantitation of the amount of antiphosphotyrosine-HRP conjugate bound to the plate.

Comparison of the assay signals obtained in the presence of compound with those of the positive and negative controls (cells incubated in the presence or absence of SCF, with no compound added), allows the degree of inhibition of c-Kit receptor tyrosine phosphorylation to be determined over a range of compound concentrations. These inhibition values were fitted to a sigmoidal dose-response inhibition curve to determine the IC50 values (i.e. the concentration of compound that inhibits SCF-induced tyrosine phosphorylation of the c-Kit protein by 50%).

The EXAMPLES of this invention reduced the ability of Kit to phosphorylate poly(Glu:Tyr) in the above assay, thus demonstrating direct inhibition of the c-Kit receptor tyrosine kinase activity. IC₅₀ values in this assay for the EXAMPLES described below were between 30 nM and 15 μM. IC₅₀ values in this assay for COMPOUNDS 1-6 were greater than 15 μM.

### EXPERIMENTAL

The EXAMPLES of the present invention were prepared according to the following procedures:

Referring to the scheme shown below for EXAMPLE 1, reaction of aminothiophene 1 with aldehydes under reducing conditions affords secondary amines such as compound 2—for example, in the presence of a mixture of triethylsilane and trifluoroacetic acid, or other reagents such as (but not limited to) sodium cyanoborohydride, sodium triacetoxyborohydride, sodium borohydride and hydrogen. Saponification of the resulting ester then gives carboxylic acid of type 3. Compounds such as 3 may then be reacted with amines in the presence of an activating agent to give carboxamides such as EXAMPLE 1.

In the section below, the following abbreviations are used: Me for methyl, Et for ethyl, Ph for phenyl, iPr for isopropyl, Bn for benzyl, THF for tetrahydrofuran, DMF for dimethylformamide, AcOH for acetic acid, EDC for 1-(3-dimethylaminopropyl)-3-ethylcarbodiimide hydrochloride, HOAt for 1-hydroxy-7-azabenzotriazole, PCC for pyridinium chlorochcomate, DMEDA for N,N-dimethylethylenediamine, EtOAc for ethyl acetate, MeCN for acetonitrile, Boc for tertbutyloxycarbnoyl, DMSO for dimethylsulfoxide, DCM for dichloromethane, Ts for toluenesulfonyl, TFA for trifluoroacetic acid, MS for mass spectroscopy, ES for electrospray, rt for room temperature, min for minute, and h for hour.

### Example 1

### N-1,4-Dioxaspiro[4.5]dec-8-yl-3-[(quinolin-4-ylmethyl)amino]thiophene-2-carboxamide

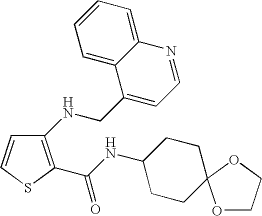

EXAMPLE 1 was prepared by the following procedure:

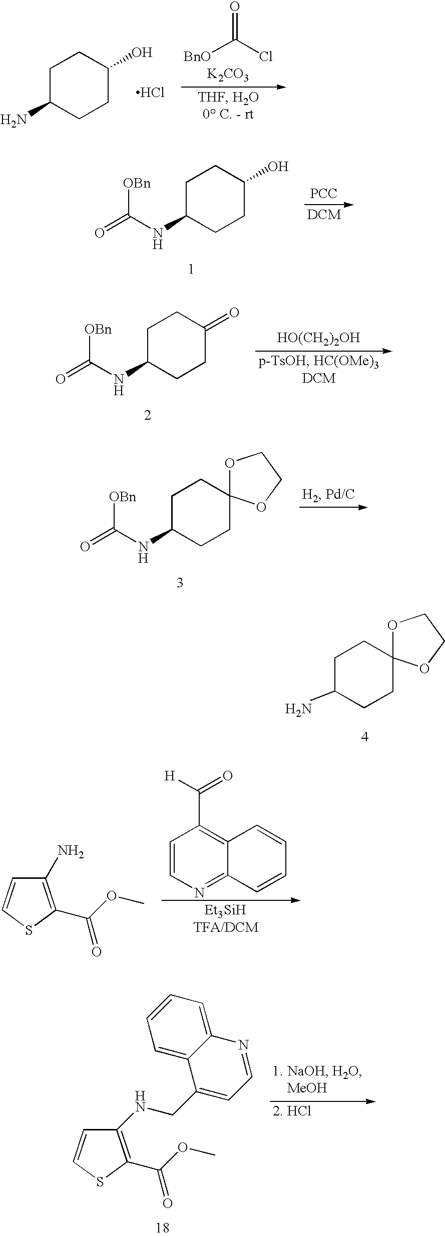

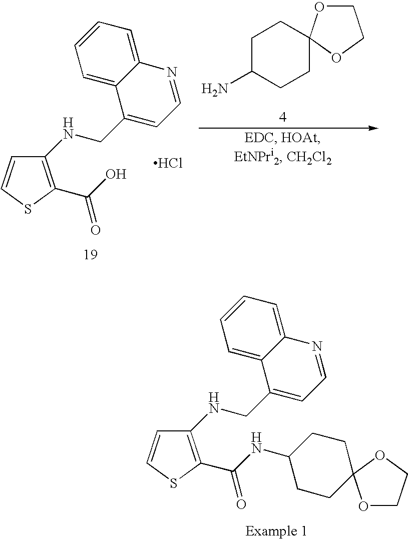

### Example 1

Part 1:

Benzyl 4-hydroxycyclohexylcarbamate (1): A mixture of trans-4-aminocyclohexanol hydrochloride (12.13 g, 80 mmol) and K₂CO₃ (24.32 g, 176 mmol) in THF (160 mL) and water (320 mL) was cooled to 0° C. A solution of benzyl chloroformate (12.4 mL, 88 mmol) in THF (16 mL) was added drop-wise. The mixture was then stirred at rt for 30 min. The mixture was then extracted with ether (200 mL) and the organic phase was washed with brine (150 mL), dried over MgSO₄, filtered, and concentrated in vacuo. The crude product was purified by recrystallization from ether to afford benzyl 4-hydroxycyclohexylcarbamate as a white solid. ¹H NMR (CDCl₃, 400 MHz): δ 1.15-1.25 (m, 2H), 1.35-1.45 (m, 2H), 1.96-2.05 (m, 4H), 3.46-3.53 (m, 1H), 3.58-3.64 (m, 1H), 4.56 (brs, 1H), 5.08 (s, 2H), 7.30-7.36 (m, 5H). MS (ES+): m/z 250 [M+1].

Part 2:

Benzyl 4-oxocyclohexylcarbamate (2): To a solution of benzyl 4-hydroxycyclohexylcarbamate (9.97 g, 40.0 mmol) in CH₂Cl₂ (190 mL) was added PCC (21.90 g, 101.6 mmol) in portions. The suspension was stirred at rt for 16 h and then filtered through a Celite pad. The filtrate was concentrated and the residue was purified by column chromatography (5% MeOH in CH₂Cl₂) to yield benzyl 4-oxocyclohexylcarbamate. ¹H NMR (CDCl₃, 400 MHz): δ 1.59-1.75 (m, 2H), 2.24 (m, 2H), 2.38-2.47 (m, 4H), 3.95-4.05 (m, 1H), 4.78 (brs, 1H), 5.12 (s, 2H), 7.32-7.40 (m, 5H). MS (ES+): m/z 248 [M+1].

Part 3:

Benzyl 1,4-dioxaspiro[4.5]dec-8-ylcarbamate (3): To a solution of benzyl 4-oxocyclohexylcarbamate (495 mg, 2.0 mmol), ethylene glycol (248 mg, 4.0 mmol) and HC(OMe)₃ (0.44 mL, 4.0 mmol) in dichloromethane (5 mL) was added p-TsOH.H₂O (19 mg, 0.1 mmol). The mixture was stirred at rt overnight. The reaction was concentrated in vacuo and the residue was purified by silica gel chromatography (1% MeOH in CH₂Cl₂) to yield benzyl 1,4-dioxaspiro[4.5]dec-8-ylcarbamate. ¹H NMR (CDCl₃, 400 MHz): δ 1.45-1.54 (m, 2H), 1.60-1.68 (m, 2H), 1.70-1.77 (m, 2H), 1.92-1.99 (m, 2H), 3.58-3.62 (m, 1H), 3.94 (s, 4H), 4.63 (brs, 1H), 5.09 (s, 2H), 7.30-7.36 (m, 5H). MS (ES+): m/z 292 [M+1].

Part 4:

1,4-Dioxaspiro[4.5]dec-8-ylamine (4): To a solution of benzyl 1,4-dioxaspiro[4.5]dec-8-ylcarbamate (475 mg, 1.6 mmol) in methanol (8 mL) was added 10% Pd/C (50 mg). The mixture was then evacuated and backfilled with N₂ atmosphere (2×) and then evacuated and backfilled with H₂ atmosphere (2×). The reaction was then stirred at rt overnight. The catalyst was removed by filtration through a Celite pad and the filtrate was concentrated to dryness to yield crude 1,4-dioxaspiro[4.5]dec-8-ylamine, which was used directly in Part 7 without further purification.

Part 5:

Methyl 3-[(quinolin-4-ylmethyl)amino]thiophene-2-carboxylate (18): A mixture of 3-amino-thiophene-2-carboxylic acid methyl ester (5 g, 31.8 mmol) and 4-quinoline-carboxaldehyde (5.25 g, 33.4 mmol) in TFA/CH₂Cl₂ (75 mL, 75 mL) was heated at 50° C. for 3.5 h. The solution was cooled in an ice bath and triethylsilane (10.2 mL, 63.6 mmol) was added drop-wise over 5 min. The reaction mixture was stirred at 50° C. for 3.5 h, cooled to rt, and 500 mL of CH₂Cl₂ was added. The reaction mixture was basified with 10N NaOH (pH 6-7) followed by sat. NaHCO₃ (pH 8). The CH₂Cl₂ layer was separated and the aqueous layer was extracted with CH₂Cl₂ (2×100 mL). The organic extracts were combined, washed with brine, dried over anhydrous sodium sulfate, filtered, and concentrated in vacuo to yield the crude product, which was triturated with hexane to give pure methyl 3-[(quinolin-4-ylmethyl)amino]thiophene-2-carboxylate as white solid. MS (ES): m/z 298.55 (100) [MH⁺]; ¹H-NMR (400 MHz/CDCl₃): δ 3.87 (s, 3H), 5.00 (d, J=4.0 Hz, 2H), 6.48 (d, J=5.6 Hz, 1H), 7.30 (d, J=5.6 Hz, 1H), 7.36 (m, 1H), 7.41 (d, J=4.4 Hz, 1H), 7.62 (t, J=8.0 Hz, 1H), 7.76 (t, J=9.6 Hz, 1H), 8.01 (d, J=8.0 Hz, 1H), 8.17 (d, J=8.4 Hz, 1H), 8.86 (d, J=4.4 Hz, 1H).

Part 6:

3-[(Quinolin-4-ylmethyl)amino]thiophene-2-carboxylic acid hydrochloride (19): To a suspension of 3-[(quinolin-4-ylmethyl)-amino]-thiophene-2carboxylic acid methyl ester (5.00 g, 16.8 mmol) in a mixture of H₂O (20 mL) and MeOH (250 mL) was added solid NaOH (6.7 g, 167.5 mmol). The reaction mixture was stirred at reflux for 5 h. The organic solvent was evaporated under reduced pressure and water (60 mL) was added to dissolve the residue. The aqueous solution was washed with ethyl acetate (50 mL) and then acidified with 1N HCl to pH 5-6. 3-[(Quinolin-4-ylmethyl)amino]thiophene-2-carboxylic acid hydrochloride precipitated from solution and was collected by filtration. ¹HNMR (400 MHz/DMSO-d₆): δ 5.07 (s, 2H), 6.71 (d, J=4.0 Hz, 1H), 7.36 (d, J=4.0 Hz, 1H), 7.43 (m, 1H), 7.58 (d, J=4.0 Hz, 1H), 7.67 (t, J=8.4 Hz, 1H), 7.80 (t, J=7.2 Hz, 1H), 8.06 (d, J=8.4 Hz, 1H), 8.22 (d, J=8.4 Hz, 1H), 8.83 (d, J=3.6 Hz, 1H), 12.30 (s, 1H). MS (ES+): 285[MH⁺].

Part 7:

N-1,4-Dioxaspiro[4.5]dec-8-yl-3-[(quinolin-4-ylmethyl)amino]thiophene-2-carboxamide: To a suspension of 3-[(quinolin-4-ylmethyl)amino]thiophene-2-carboxylic acid hydrochloride (96 mg, 0.3 mmol) and 1,4-dioxaspiro[4.5]dec-8-ylamine (47 mg, 0.3 mmol) in dichloromethane (5 mL) was added EDC (72 mg, 0.38 mmol), HOAt (0.5M solution in DMF, 0.18 mL, 0.09 mmol) and i-Pr₂NEt (0.16 mL). The mixture was stirred at rt for 18 h. The reaction was concentrated in vacuo and the residue was purified by silica gel chromatography (5-10% MeOH in dichloromethane) to afford EXAMPLE 1. ¹H NMR (CDCl₃, 400 MHz): δ 1.56-1.82 (m, 8H), 3.95-4.05 (m, 5H), 4.96 (d, J=6.4 Hz, 2H), 5.27 (d, br, J=8.0 Hz, 1H), 6.51 (d, J=5.2 Hz, 1H), 7.14 (d, J=4.8 Hz, 1H), 7.46 (d, J=4.0 Hz, 1H), 7.61 (dt, J=0.8, 8.4 Hz, 1H), 7.75 (dt, J=1.6, 8.8 Hz, 1H), 7.96 (d, br, J=5.6 Hz, 1H), 8.02 (d, J=8.8 Hz, 1H), 8.16 (d, J=8.4 Hz, 1H), 8.85 (d, J=4.4 Hz, 1H). MS (ES+): m/z 424 [M+1].

The following analogues were prepared according to the procedure described above for EXAMPLE 1, using 2-mercaptoethanol instead of ethylene glycol.

### Example 2

### cis-N-1-Oxa-4-thiaspiro[4.5]dec-8-yl-3-[(quinolin-4-ylmethyl)amino]thiophene-2-carboxamide: MS (ES+) 440 [M+1]

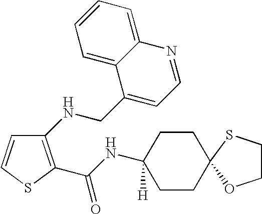

### Example 3

### trans-N-1-Oxa-4-thiaspiro[4.5]dec-8-yl-3-[(quinolin-4-ylmethyl)amino]thiophene-2-carboxamide: MS (ES+) 440 [M+1]

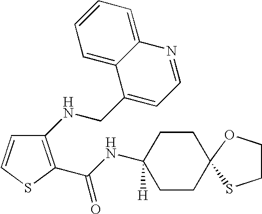

### Example 4

### N-1,4-Dioxaspiro[4.5]dec-8-yl-4-methyl-3-[(quinolin-4-ylmethyl)amino]thiophene-2-carboxamide

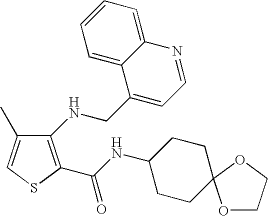

EXAMPLE 4 was prepared according to the procedure described for EXAMPLE 1 using 3-amino-4-methyl-thiophene-2-carboxylic acid methyl ester instead of 3-amino-thiophene-2-carboxylic acid methyl ester: MS (ES+) 438 [M+1].

### Example 5

### cis-N-(1,4-Dimethyl-5-oxo-1,4-diazaspiro[5.5]undec-9-yl)-3-[(quinolin-4-ylmethyl)amino]thiophene-2-carboxamide

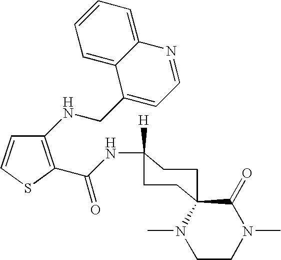

EXAMPLE 5 was prepared according to the procedure described for EXAMPLE 1 using 9-amino-1,4-dimethyl-1,4-diazaspiro[5.5]undecan-5-one (compound 7 in the procedure below) instead of 1,4-dioxaspiro[4.5]dec-8-ylamine. MS (ES+) 478 [M+1].

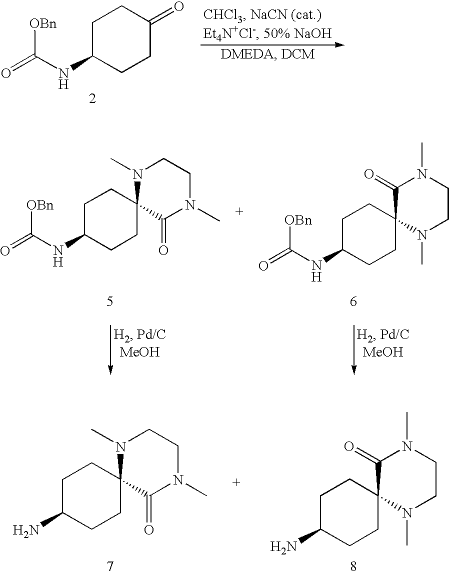

Part 1:

Benzyl 1,4-dimethyl-5-oxo-1,4-diazaspiro[5.5]undec-9-ylcarbamate (5, 6): A solution of benzyl 4-oxocyclohexylcarbamate (74 mg, 0.30 mmol), N,N-dimethylethylenediamine (40 mg, 0.45 mmol), CHCl₃ (54 mg, 0.45 mmol), NaCN (0.9 mg, 0.02 mmol), Et₄N⁺Cl⁻ (1.5 mg, 9.0 μmol) in dichloromethane was cooled to 0° C. and 50% aqueous NaOH (0.5 mL) was added. The mixture was stirred vigorously at rt for 24 h and then poured into a separation funnel. Dichloromethane and water were added. The organic phase was removed and the aqueous solution was extracted with dichloromethane. The combined organic extracts were dried over MgSO₄, filtered, and concentrated in vacuo to a crude mixture of spiro compounds 5 and 6. The crude mixture was purified by silica gel chromatography (1-5% MeOH in DCM) to yield benzyl 1,4-dimethyl-5-oxo-1,4-diazaspiro[5.5]undec-9-ylcarbamate. Analytical data for cis isomer (5): ¹H NMR (CDCl₃, 400 MHz): δ 1.39-1.47 (m, 2H), 1.80-1.88 (m, 2H), 1.89-2.01 (m, 4H), 2.42 (s, 3H), 2.92 (s, 3H), 3.13 (t, J=6.0 Hz, 2H), 3.36 (t, J=6.0 Hz, 2H), 3.57-3.62 (m, 1H), 4.62 (brs, 1H), 5.09 (s, 2H), 7.31-7.37 (m, 5H). MS (ES+): m/z 346 [M+1]. Analytical data for trans isomer (6): ¹H NMR (CDCl₃, 400 MHz): δ 1.70-1.77 (m, 4H), 1.84-1.90 (m, 2H), 1.97-2.02 (m, 2H), 2.42 (s, 3H), 2.91 (s, 3H), 3.10 (t, J=5.6 Hz, 2H), 3.36 (t, J=5.6 Hz, 2H), 3.75-3.80 (m, 1H), 5.09 (s, 2H), 5.12 (brs, 1H), 7.30-7.36 (m, 5H). MS (ES+): m/z 346 [M+1].

Part 2:

9-Amino-1,4-dimethyl-1,4-diazaspiro[5.5]undecan-5-one (7): To a solution of benzyl 1,4-dimethyl-5-oxo-1,4-diazaspiro[5.5]undec-9-ylcarbamate (5 mg, 0.014 mmol) in methanol (1 mL) was added 10% Pd/C (5 mg, 4.6 μmol). The solution was then deoxygenated and back filled with N₂ atmosphere (repeated 2×). The solution was then degassed and back filled with H₂ atmosphere (repeated 2×). The mixture was then stirred under H₂ atmosphere at rt overnight. The catalyst was removed by filtration through a Celite pad and the filtrate was concentrated to dryness to give the crude 9-amino-1,4-dimethyl-1,4-diazaspiro[5.5]undecan-5-one, which was used directly in the next step without purification.

### Example 6

### trans-N-(1,4-Dimethyl-5-oxo-1,4-diazaspiro[5.5]undec-9-yl)-3-[(quinolin-4-ylmethyl)amino]thiophene-2-carboxamide

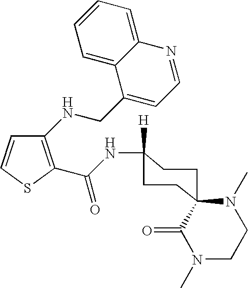

EXAMPLE 6 was prepared according to the procedure described for EXAMPLE 1 using 9-amino-1,4-dimethyl-1,4-diazaspiro[5.5]undecan-5-one (compound 8 in the procedure above) instead of 1,4-dioxaspiro[4.5]dec-8-ylamine. MS (ES+) 478 [M+1].

### Example 7

### cis-N-(3′-Oxo-3′,4′-dihydro-1′H-spiro[cyclohexane-1,2′-quinoxalin]4-yl)-3-[(quinolin-4-ylmethyl)amino]thiophene-2-carboxamide

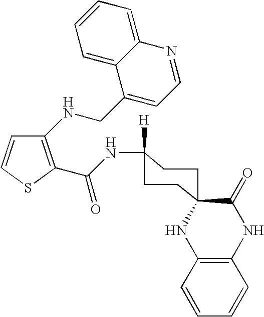

EXAMPLE 7 was prepared according to the procedure described for EXAMPLE 5 using 1,2-phenylenediamine instead of N,N-dimethylethylenediamine. MS (ES+) 498 [M+1].

### Example 8

### trans-N-(3′-Oxo-3′,4′-dihydro-1′H-spiro[cyclohexane-1,2′-quinoxalin]-4-yl)-3-[(quinolin-4-ylmethyl)amino]thiophene-2-carboxamide

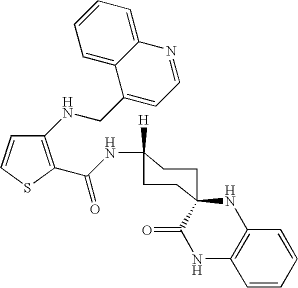

EXAMPLE 8 was prepared according to the procedure described for EXAMPLE 6 using 1,2-phenylenediamine instead of N,N-dimethylethylenediamine. MS (ES+) 498 [M+1].

### Compound 1

### cis-N-(4-Methyl-5-oxo-1,4-diazaspiro[5.5]undec-9-yl)-3-[(quinolin-4-ylmethyl)amino]thiophene-2-carboxamide

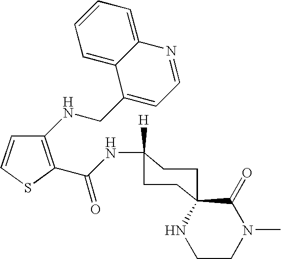

COMPOUND 1 was prepared according to the procedure described for EXAMPLE 6 using N-methylethylenediamine instead of N,N-dimethylethylenediamine. MS (ES+) 464 [M+1].

### Example 9

### N-(3-Oxo-2-azaspiro[4.5]dec-8-yl)-3-[(quinolin-4-ylmethyl)amino]thiophene-2-carboxamide

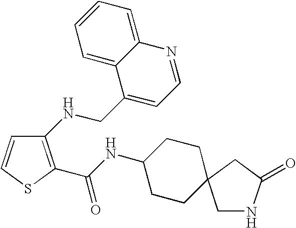

EXAMPLE 9 was prepared according to the procedure described for EXAMPLE 1 using 8-amino-2-azaspiro[4.5]decan-3-one (compound 12 in the procedure below) instead of 1,4-dioxaspiro[4.5]dec-8-ylamine. MS (ES+) 435 [M+1].

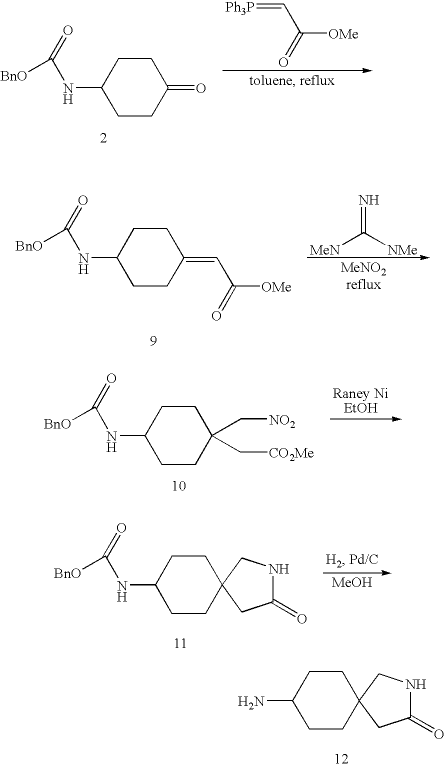

Part 1:

Methyl (4-benzyloxycarbonylaminocyclohexylidene)acetate (9): A solution of benzyl 4-oxocyclohexylcarbamate (1.24 g, 5.1 mmol) and methyl (triphenylphosphoranylidene)acetate (2.09 g, 6.3 mmol) in toluene (10 mL) was refluxed for 18 h. The solvent was removed in vacuo and the residue was purified by silica gel chromatography (1-5% MeOH in CH₂Cl₂) to afford methyl (4-benzyloxycarbonylaminocyclohexylidene)acetate. ¹H NMR (CDCl₃, 400 MHz): δ 1.28-1.41 (m, 2H), 2.05-2.30 (m, 5H), 3.60-3.64 (m, 1H), 3.09 (s, 3H), 3.76-3.82 (m, 1H), 4.62-4.68 (m, 1H), 5.10 (s, 2H), 5.65 (s, 1H), 7.31-7.37 (m, 5H). MS (ES+): m/z 304 [M+1].

Part 2:

Methyl (4-benzyloxycarbonylamino-1-nitromethylcyclohexyl)acetate (10): To a solution of methyl (4-benzyloxycarbonyl aminocyclohexylidene)acetate (303 mg, 1.0 mmol) in nitromethane (5 mL) was added 1,1,3,3-tetramethylguanidine (46 μL). The mixture was refluxed for 24 h. The solvent was removed in vacuo and the residue was purified by silica gel chromatography (1% MeOH in CH₂Cl₂) to yield methyl (4-benzyloxycarbonylamino-1-nitromethylcyclo hexyl)acetate. ¹H NMR (CDCl₃, 400 MHz): δ 1.33-2.12 (m, 8H), 2.61 (s, 2H), 3.52-3.55 (m, 1H), 3.68 (s, 2H), 3.74 (s, 3H), 4.66-4.69 (m, 1H), 5.09 (s, 2H), 7.31-7.37 (m, 5H).

Part 3:

8-Amino-2-azaspiro[4.5]decan-3-one (12): Nitro compound methyl (4-benzyloxycarbonylamino-1-nitromethylcyclohexyl)acetate (130 mg, 0.36 mmol) was dissolved in EtOH (5 mL) and Raney Ni (50% slurry in water, 3 mL) was added under N₂ atmosphere. The resulting suspension was stirred at rt for 72 h. The mixture was filtered through a Celite pad and solvent was removed in vacuo yield benzyl (3-oxo-2-azaspiro[4.5]dec-8-yl)carbamate (11) as an oil. Crude benzyl (3-oxo-2-azaspiro[4.5]dec-8-yl)carbamate was then dissolved in methanol (10 mL) and 10% Pd/C (100 mg) was added. The solution was then deoxygenated and back filled with N₂ atmosphere (repeated 2×). The solution was then degassed and back filled with H₂ atmosphere (repeated 2×). The mixture was then stirred under H₂ atmosphere at rt overnight. The mixture was filtered through a Celite pad and solvent was removed in vacuo yield crude 8-amino-2-azaspiro[4.5]decan-3-one, which was used directly in the coupling reaction without purification.

### Example 10

### N-(2-Methyl-3-oxo-1,2-diazaspiro[4.5]dec-8-yl)-3-[(quinolin-4-ylmethyl)amino]thiophene-2-carboxamide

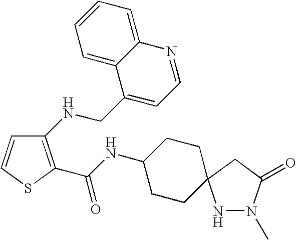

EXAMPLE 10 was prepared according to the procedure described for EXAMPLE 1 using 8-amino-2-methyl-1,2-diazaspiro[4.5]decan-3-one (compound 14 in the procedure below) instead of 1,4-dioxaspiro[4.5]dec-8-ylamine. MS (ES+) 450 [M+1].

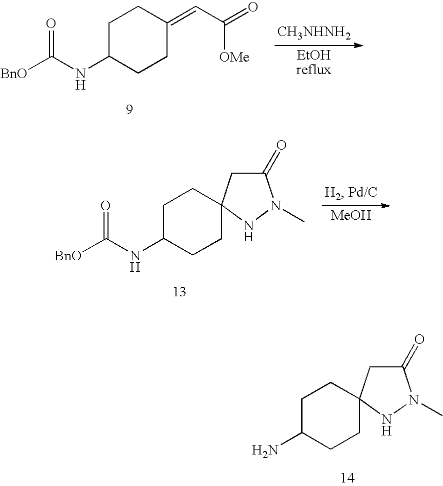

Part 1:

Benzyl 2-methyl-3-oxo-1,2-diazaspiro[4.5]dec-8-ylcarbamate (13): A solution of methylhydrazine (15.2 mg, 0.33 mmol) and ester 9 (91 mg, 0.30 mmol) in EtOH (1 mL) was refluxed for 15 h. The solvent was removed in vacuo and the residue was purified by silica gel chromatography (5% MeOH in CH₂Cl₂) to yield benzyl 2-methyl-3-oxo-1,2-diazaspiro[4.5]dec-8-ylcarbamate. ¹H NMR (CDCl₃, 400 MHz): δ 1.53-1.82 (m, 6H), 2.00-2.04 (m, 2H), 2.40 (s, 2H), 3.01 (s, 3H), 3.52-3.60 (m, 1H), 4.23 (brs, 1H), 4.62-4.69 (m, 1H), 5.09 (s, 2H), 7.30-7.44 (m, 5H). MS (ES+): m/z 318 [M+1].

Part 2:

8-Amino-2-methyl-1,2-diazaspiro[4.5]decan-3-one (14): To a solution of benzyl 2-methyl-3-oxo-1,2-diazaspiro[4.5]dec-8-ylcarbamate (74 mg, 0.23 mmol) in methanol (5 mL) was added 10% Pd/C (150 mg, 0.14 mmol). The solution was then deoxygenated and back filled with N₂ atmosphere (repeated 2×). The solution was then degassed and back filled with H₂ atmosphere (repeated 2×). The mixture was then stirred under H₂ atmosphere at rt overnight. The catalyst was removed by filtration through a Celite pad and the filtrate was concentrated in vacuo to yield crude 8-amino-2-methyl-1,2-diazaspiro[4.5]decan-3-one, which was used directly in the next step without purification.

### Example 11

### trans-N-(2-Benzyl-3-oxo-1,2-diazaspiro[4.5]dec-8-yl)-3-[(quinolin-4-ylmethyl)amino]thiophene-2-carboxamide

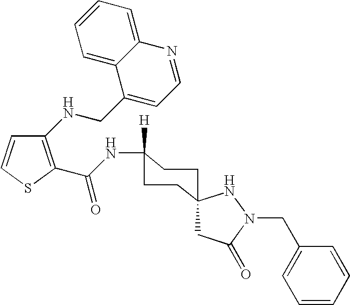

EXAMPLE 11 was prepared according to the procedure described for EXAMPLE 1 using 8-amino-2-benzyl-1,2-diazaspiro[4.5]decan-3-one (prepared according the procedure described above for compound 14 using benzylhydrazine instead of methylhydrazine) instead of 1,4-dioxaspiro[4.5]dec-8-ylamine. MS (ES+) 526 [M+1].

### Compound 2

### cis-N-(2-Benzyl-3-oxo-1,2-diazaspiro[4.5]dec-8-yl)-3-[(quinolin-4-ylmethyl)amino]thiophene-2-carboxamide

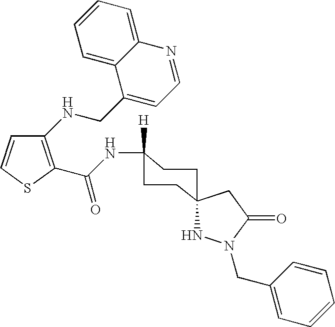

COMPOUND 2 was prepared according to the procedure described for EXAMPLE 1 using 8-amino-2-benzyl-1,2-diazaspiro[4.5]decan-3-one (prepared according the procedure described above for compound 14 using benzylhydrazine instead of methythydrazine) instead of 1,4-dioxaspiro[4.5]dec-8-ylamine. MS (ES+) 526 [M+1].

### Compound 3

### N-(3-Benzyl-2,4-dioxo-1,3-diazaspiro[4.5]dec-8-yl)-3-[(quinolin-4-ylmethyl)amino]thiophene-2-carboxamide

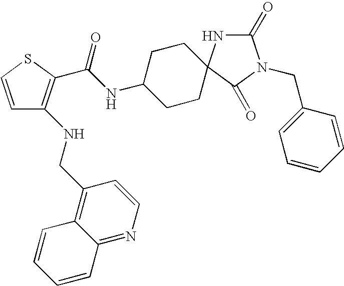

COMPOUND 3 was prepared according to the procedure described for EXAMPLE 1 using 8-amino-3-benzyl-1,3-diazaspiro[4.5]decane-2,4-dione (compound 17 in the procedure below) instead of 1,4-dioxaspiro[4.5]dec-8-ylamine. MS (ES+) 435 [M+1].

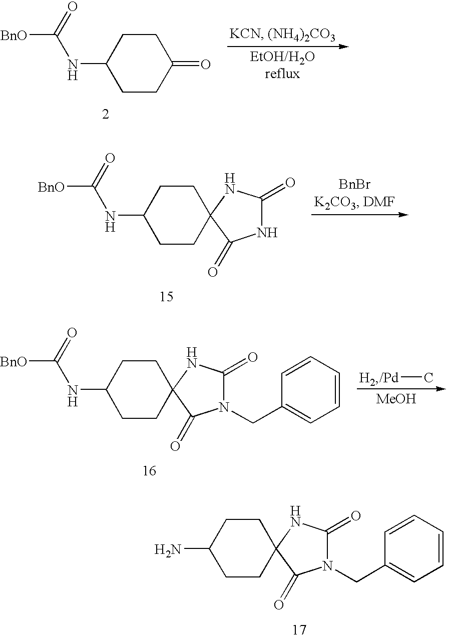

Part 1:

Benzyl 2,4-dioxo-1,3-diazaspiro[4.5]dec-8-ylcarbamate (15): A mixture of ketone 2 (800 mg, 3.2 mmol), KCN (314 mg, 4.8 mmol), and (NH₄)₂CO₃ (930 mg, 9.7 mmol) in a 1:1 solution of ethanol/water (20 mL) was heated to reflux. After 18 h the reaction was cooled to rt and the solvent was reduced in vacuo until precipitation occurred. The solution was filtered and benzyl 2,4-dioxo-1,3-diazaspiro[4.5]dec-8-ylcarbamate was collected as a white solid. ¹H NMR (400 MHz/CDCl₃): δ 1.22-1.40 (m, 2H), 1.76-1.80 (m, 2H), 1.90-2.2 (m, 4H), 3.53-3.65 (m, 1H), 5.10 (s, 2H), 7.25-7.40 (m, 5H). MS (ES+): 318[M+1].

Part 2:

Benzyl 3-benzyl-2,4-dioxo-1,3-diazaspiro[4.5]dec-8-ylcarbamate (16): A mixture of benzyl 2,4-dioxo-1,3-diazaspiro[4.5]dec-8-ylcarbamate (80 mg, 0.25 mmol), benzyl bromide (33 μL, 0.28 mmol), and K₂CO₃ (34.6 mg, 0.25 mmol) in DMF was stirred at rt for 24 h. The reaction mixture was then partitioned between ethyl acetate and water. The organic layer was concentrated in vacuo and the residue was washed with a minimum amount of MeOH and ethyl acetate to give pure benzyl 3-benzyl-2,4-dioxo-1,3-diazaspiro[4.5]dec-8-ylcarbamate. ¹H NMR (400 MHz/DCl₃): δ 1.23-1.29 (m, 2H), 1.67 (d, J=13.7 Hz, 2H), 1.99 (t, J=14.8 Hz, 2H), 2.12 (d, J=11.6 Hz, 2H), 3.54-3.67 (m, 1H), 4.64 (s, 3H), 5.10 (s, 2H), 6.49 (s, 1H), 7.31-7.35 (m, 10H). MS (ES+): 408 [M+1].

Part 3:

8-Amino-3-benzyl-1,3-diazaspiro[4.5]decane-2,4-dione (17): Benzyl 3-benzyl-2,4-dioxo-1,3-diazaspiro[4.5]dec-8-ylcarbamate was dissolved in MeOH (13 mg, 0.032 mmol) and 10% Pd/C (10 mg, 9 μmol) was added. The solution was then deoxygenated and back filled with N₂ atmosphere (repeated 2×). The solution was then degassed and back filled with H₂ atmosphere (repeated 2×). The mixture was then stirred under H₂ atmosphere at rt overnight. The mixture was filtered through a Celite pad and solvent was removed in vacuo yield pure 8-amino-3-benzyl-1,3-diazaspiro[4.5]decane-2,4-dione. MS (ES+): 274 [M+1].

### Example 12

### trans-N-(2,4-Dioxo-1,3-diazaspiro[4.5]dec-8-yl)-3-[(quinolin-4-ylmethyl)amino]thiophene-2-carboxamide: MS (ES+) 450 [M+1]

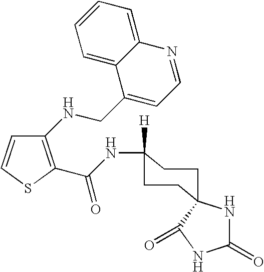

EXAMPLE 12 was prepared using 3-[(Quinolin-4-ylmethyl)amino]thiophene-2-carboxylic acid hydrochloride (19, EXAMPLE 1) and the appropriate amine (prepared according to the procedure described above for COMPOUND 3 omitting the alkylation step of part 2), according to the procedure described for EXAMPLE 1.

The following analogues were prepared using 3-[(Quinolin-4-ylmethyl)amino]thiophene-2-carboxylic acid hydrochloride (19, EXAMPLE 1) and the appropriate amine (prepared according to the procedure described above for COMPOUND 3 using the appropriate alkylating agent in part 2), according to the procedure described for EXAMPLE 1.

### Compound 4

### N-(2,4-Dioxo-1,3-diazaspiro[4.5]dec-8-yl)-3-[(quinolin-4-ylmethyl)amino]thiophene-2-carboxamide: MS (ES+) 450 [M+1]

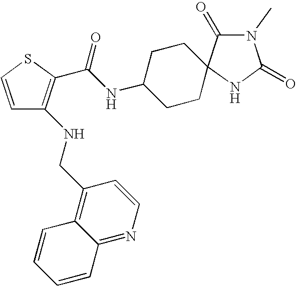

### Example 13

### N-(3-Methyl-2,4-dioxo-1,3-diazaspiro[4.5]dec-8-yl)-3-[(quinolin-4-ylmethyl)amino]thiophene-2-carboxamide: MS (ES+) 464 [M+1]

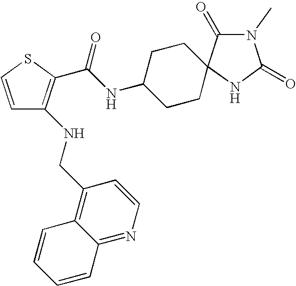

### Example 14

### N-[3-(3-Methylbutyl)-2,4-dioxo-1,3-diazaspiro[4.5]dec-8-yl]-3-[(quinolin-4-ylmethyl)amino]thiophene-2-carboxamide: MS (ES+) 520 [M+1]

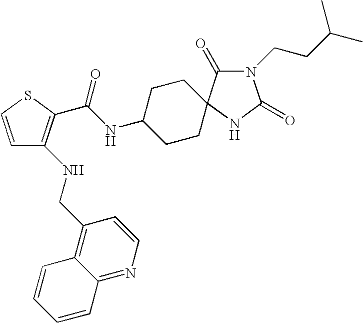

### Compound 5

### N-[3-(3-Methylbutyl)-2,4-dioxo-1,3-diazaspiro[4.5]dec-8-yl]-4-methyl-3-[(quinolin-4-ylmethyl)amino]thiophene-2-carboxamide

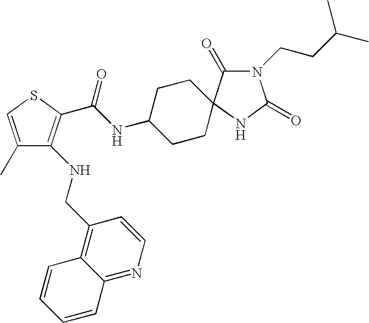

COMPOUND 5 was prepared using 3-[(Quinolin-4-ylmethyl)amino]thiophene-2-carboxylic acid hydrochloride (prepared according to the procedure described for EXAMPLE 1 using 3-amino-4-methyl-thiophene-2-carboxylic acid methyl ester instead of 3-amino-thiophene-2-carboxylic acid methyl ester) and 8-amino-3-(3-methylbutyl)-1,3-diazaspiro[4.5]decane-2,4-dione (prepared according to the procedure described for COMPOUND 3 using the appropriate alkylating agent in part 2), according to the procedure described for EXAMPLE 1. MS (ES+) 438 [M+1].

### Example 15

### N-(1′-Methyl-2-oxo-1,2-dihydrospiro[indole-3,4′-piperidin]-5-yl)-3-[(quinolin-4-ylmethyl)amino]thiophene-2-carboxamide

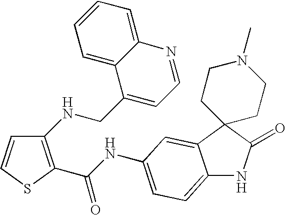

EXAMPLE 15 was prepared by the following procedure:

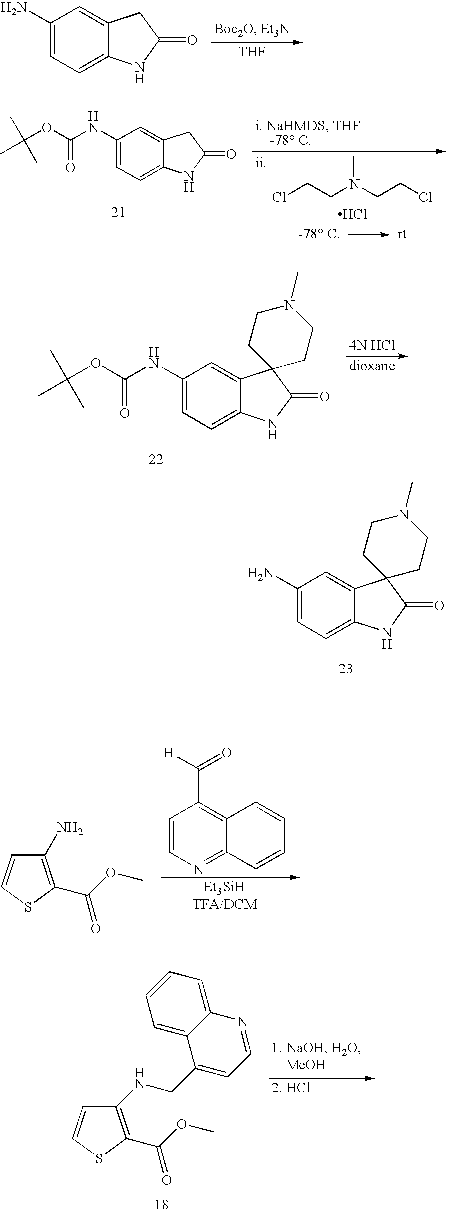

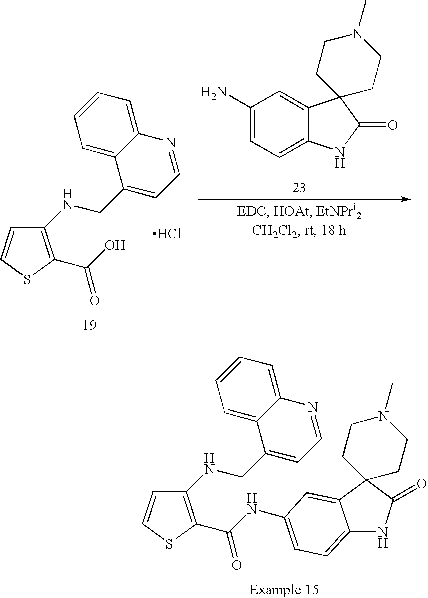

### Example 15

Part 1:

tert-Butyl 2-oxo-2,3-dihydro-1H-indol-5-ylcarbamate (21): To a mixture of 5-amino-1,3-dihydro-2H-indol-2-one (148 mg, 11.0 mmol), di-tert-butyl dicarbonate (262 mg, 1.2 mmol), and Et₃N (279 μL, 2.0 mmol) was added dry THF (5 mL). The suspension was then stirred at rt for 24 h. The reaction was concentrated in vacuo to yield tert-butyl 2-oxo-2,3-dihydro-1H-indol-5-ylcarbamate as a brown solid. ¹H NMR (CDCl₃, 400 MHz): δ 1.52 (s, 9H), 3.52 (s, 2H), 6.38 (brs, 1H), 6.76 (d, J=8.0 Hz, 1H), 7.07 (dd, J=2.4, 8.4 Hz, 1H), 7.26 (d, J=2.4 Hz, 1H), 7.42 (brs, 1H). MS (ES+): m/z 249 [M+1].

Part 2:

tert-Butyl 1′-methyl-2-oxo-1,2-dihydrospiro[indole-3,4′-piperidin]-5-ylcarbamate (22): A solution of tert-butyl 2-oxo-2,3-dihydro-1H-indol-5-ylcarbamate (248 mg, 1.0 mmol) in THF (3 mL) was cooled to −78° C. and a 1M solution of sodium hexamethyldisilazide in THF (6 mL, 6.0 mmol) was added drop-wise. After stirring at −78° C. for 30 min, N,N-bis(2-chloroethyl)-N-methylamine hydrochloride (193 mg, 1.0 mmol) was added. The reaction mixture was stirred at −78° C. for 30 min and then at rt for 2 days. The reaction was quenched with water (2 mL) and the mixture was extracted with EtOAc (3×50 mL). The organic extracts were combined, dried over MgSO₄, filtered, and concentrated in vacuo. The residue was purified by silica gel chromatography (10% MeOH in dichloromethane) to yield tert-butyl 1′-methyl-2-oxo-1,2-dihydrospiro[indole-3,4′-piperidin]-5-ylcarbamate. ¹H NMR (CDCl₃, 400 MHz): δ 1.51 (s, 9H), 1.88-1.93 (m, 2H), 2.52-2.62 (m, 2H), 2.72 (s, 3H), 3.14-3.17 (m, 2H), 3.44-3.50 (m, 2H), 6.64 (s, 1H), 6.84 (d, J=8.4 Hz, 1H), 7.47 (brs, 1H), 7.83 (brs, 1H). MS (ES+): m/z 332 [M+1].

Part 3:

5-Amino-1′-methylspiro[indole-3,4′-piperidin]-2(1H)-one (23): To a solution of tert-butyl 1′-methyl-2-oxo-1,2-dihydrospiro[indole-3,4′-piperidin]-5-ylcarbamate (50 mg, 0.15 mmol) in dioxane (2 mL) was added 4N HCl (1 mL). The mixture was stirred at rt for 16 h. The reaction was concentrated in vacuo and the residue was dissolved in dichloromethane (10 mL). Saturated NaHCO₃ (5 mL) was added and the mixture was stirred at rt for 1 h. The organic phase was removed and the aqueous phase was extracted with dichloromethane (2×10 mL). The organic extracts were combined, dried over MgSO₄, filtered, and concentrated in vacuo. The residue was purified by silica gel chromatography (15% MeOH in dichloromethane) to afford 5-amino-1′-methylspiro[indole-3,4′-piperidin]-2(1H)-one. ¹H NMR (CDCl₃, 400 MHz): δ 1.88-1.93 (m, 2H), 2.70-2.90 (m, 5H), 3.23-3.32 (m, 2H), 3.58-3.70 (m, 2H), 6.55 (d, J=8.0 Hz, 1H), 6.67 (d, J=8.0 Hz, 1H), 6.79 (s, 1H), 7.13 (brs, 1H). MS (ES+): m/z 232 [M+1].

Part 4:

Methyl 3-[(quinolin-4-ylmethyl)amino]thiophene-2-carboxylate (18): A mixture of 3-amino-thiophene-2-carboxylic acid methyl ester (5 g, 31.8 mmol) and 4-quinoline-carboxaldehyde (5.25 g, 33.4 mmol) in TFA/CH₂Cl₂ (75 mL, 75 mL) was heated at 50° C. for 3.5 h. The solution was cooled in an ice bath and triethylsilane (10.2 mL, 63.6 mmol) was added drop-wise over 5 min. The reaction mixture was stirred at 50° C. for 3.5 h, cooled to rt, and 500 mL of CH₂Cl₂ was added. The reaction mixture was basified with 10N NaOH (pH 6-7) followed by sat. NaHCO₃ (pH 8). The CH₂Cl₂ layer was separated and the aqueous layer was extracted with CH₂Cl₂ (2×100 mL). The organic extracts were combined, washed with brine, dried over anhydrous sodium sulfate, filtered, and concentrated in vacuo to yield the crude product, which was triturated with hexane to give pure methyl 3-[(quinolin-4-ylmethyl)amino]thiophene-2-carboxylate as white solid. MS (ES): m/z 298.55 (100) [MH⁺]; ¹H-NMR (400 MHz/CDCl₃): δ 3.87 (s, 3H), 5.00 (d, J=4.0 Hz, 2H), 6.48 (d, J=5.6 Hz, 1H), 7.30 (d, J=5.6 Hz, 1H), 7.36 (m, 1H), 7.41 (d, J=4.4 Hz, 1H), 7.62 (t, J=8.0 Hz, 1H), 7.76 (t, J=9.6 Hz, 1H), 8.01 (d, J=8.0 Hz, 1H), 8.17 (d, J=8.4 Hz, 1H), 8.86 (d, J=4.4 Hz, 1H).

Part 5:

3-[(Quinolin-4-ylmethyl)amino]thiophene-2-carboxylic acid hydrochloride (19): To a suspension of 3-[(quinolin-4-ylmethyl)-amino]-thiophene-2carboxylic acid methyl ester (5.00 g, 16.8 mmol) in a mixture of H₂O (20 mL) and MeOH (250 mL) was added solid NaOH (6.7 g, 167.5 mmol). The reaction mixture was stirred at reflux for 5 h. The organic solvent was evaporated under reduced pressure and water (60 mL) was added to dissolve the residue. The aqueous solution was washed with ethyl acetate (50 mL) and then acidified with 1N HCl to pH 5-6. 3-[(Quinolin-4-ylmethyl)amino]thiophene-2-carboxylic acid hydrochloride precipitated from solution and was collected by filtration. ¹HNMR (400 MHz/DMSO-d₆): δ 5.07 (s, 2H), 6.71 (d, J=4.0 Hz, 1H), 7.36 (d, J=4.0 Hz, 1H), 7.43 (m, 1H), 7.58 (d, J=4.0 Hz, 1H), 7.67 (t, J=8.4 Hz, 1H), 7.80 (t, J=7.2 Hz, 1H), 8.06 (d, J=8.4 Hz, 1H), 8.22 (d, J=8.4 Hz, 1H), 8.83 (d, J=3.6 Hz, 1H), 12.30 (s, 1H). MS (ES+): 285[MH⁺].

Part 6:

N-(1′-Methyl-2-oxo-1,2-dihydrospiro[indole-3,4′-piperidin]-5-yl)-3-[(quinolin-4-ylmethyl)amino]thiophene-2-carboxamide (24): To a suspension of 3-[(quinolin-4-ylmethyl)amino]thiophene-2-carboxylic acid hydrochloride (45 mg, 0.14 mmol) and 5-amino-1′-methylspiro[indole-3,4′-piperidin]-2(1H)-one (25 mg, 0.11 mmol) in dichloromethane (2 mL) was added EDC (29 mg, 0.15 mmol), HOAt (0.5M solution in DMF, 60 μL, 0.03 mmol) and i-Pr₂NEt (0.13 mL, 0.76 mmol). The mixture was stirred at rt for 18 h. The reaction was then concentrated in vacuo and the residue was purified by silica gel chromatography (5-10% MeOH in dichloro-methane) to afford EXAMPLE 15. ¹H NMR (CDCl₃, 400 MHz): δ 1.96-2.05 (m, 4H), 2.45 (s, 3H), 2.68-2.73 (m, 2H), 2.90-2.96 (m, 2H), 5.00 (d, J=6.0 Hz, 2H), 6.56 (d, J=5.6 Hz, 1H), 6.85 (d, J=8.4 Hz, 1H), 7.17 (s, 1H), 7.23 (d, J=5.2 Hz, 1H), 7.33 (dd, J=2.0, 8.4 Hz, 1H), 7.46 (d, J=4.8 Hz, 1H), 7.59-7.64 (m, 2H), 7.76 (dd, J=0.8, 8.0 Hz, 1H), 8.02-8.05 (m, 2H), 8.09 (brs, 1H), 8.17 (d, J=8.0 Hz, 1H), 8.86 (d, J=4.4 Hz, 1H). MS (ES+): m/z 498 [M+1].

### Example 16

### N-(2-Oxo-1,2,2′,3′,5′,6′-hexahydrospiro[indole-3,4′-pyran]-5-yl)-3-[(quinolin-4-ylmethyl)amino]thiophene-2-carboxamide

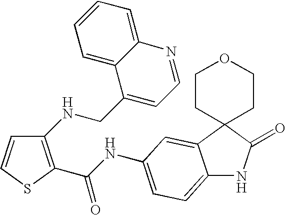

The following analogues were prepared according to the procedure described above for EXAMPLE 15, using diethylene glycol di(p-toluenesulfonate) instead of N,N-bis(2-chloroethyl)-N-methylamine hydrochloride. MS (ES+) 485 [M+1].

### Compound 6

### Benzyl 5-{[(3-[(quinolin-4-ylmethyl)amino]thien-2-yl)carbonyl]amino}-2-oxo-1,2-dihydro-1′H-spiro[indole-3,4′-piperidine]-1′-carboxylate

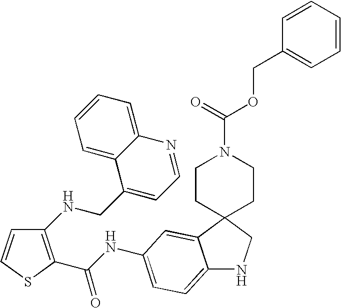

COMPOUND 6 was prepared according to the procedure described for EXAMPLE 15 using benzyl 5-amino-1,2-dihydro-1′H-spiro[indole-3,4′-piperidine]-1′-carboxylate (compound 26 in the procedure below) instead of 5-amino-1′-methylspiro[indole-3,4′-piperidin]-2(1H)-one. MS (ES+) 604 [M+1].

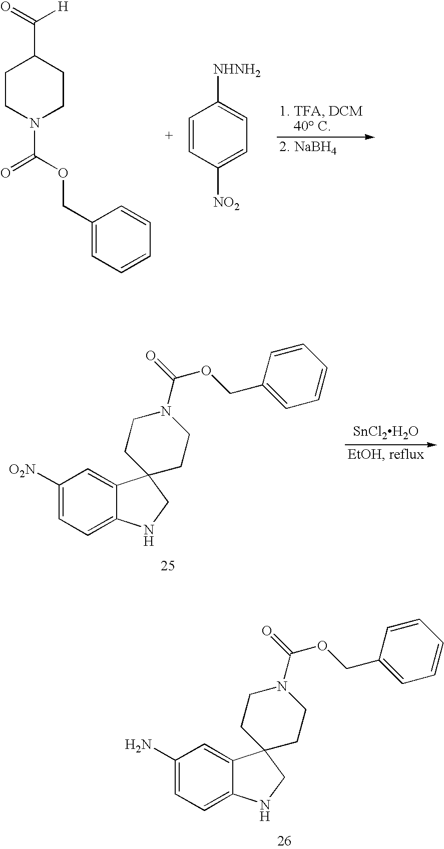

Part 1:

Benzyl 5-nitro-1,2-dihydro-1′H-spiro [indole-3,4′-piperidine]-1′-carboxylate (25): To a solution of 4-nitrophenylhydrazine (842 mg, 5.5 mmol) and TFA (5 mL) in dichloromethane (20 mL) was added benzyl 4-formylpiperidine-1-carboxylate (1.24 g, 5.0 mmol). The mixture was stirred at 40° C. for 16 h. The reaction was then cooled to rt and NaBH₄ (378 mg, 10.0 mmol) was added portion-wise over 30 min. The mixture was then stirred for an additional 30 min and then washed with 10% NH₄OH (100 mL). The organic phase was dried over MgSO₄, filtered, and concentrated in vacuo. The crude product was purified by silica gel chromatography (5% MeOH in dichloromethane) to yield benzyl 5-nitro-1,2-dihydro-1′H-spiro[indole-3,4′-piperidine]-1′-carboxylate. ¹H NMR (CDCl₃, 400 MHz): δ 1.70-1.80 (m, 2H), 1.80-1.95 (m, 2H), 2.92-3.01 (m, 2H), 3.67 (s, 2H), 4.13-4.28 (m, 2H), 4.48 (s, 1H), 5.18 (s, 2H), 6.52 (dd, J=2.0, 8.8 Hz, 1H), 7.34-7.40 (m, 5H), 7.89 (d, J=2.0 Hz, 1H), 8.04 (dt, J=2.4, 6.4 Hz, 1H). MS (ES+): m/z 368 [M+1].

Part 2:

Benzyl 5-amino-1,2-dihydro-1′H-spiro [indole-3,4′-piperidine]-1′-carboxylate (26): A suspension of benzyl 5-nitro-1,2-dihydro-1′H-spiro[indole-3,4′-piperidine]-1′-carboxylate (735 mg, 2.0 mmol) and SnCl₂.2H₂O (4.51 g, 20.0 mmol) in EtOH (30 mL) was stirred at reflux for 6 h. After cooling to room temperature, sat. NaHCO₃ (aq.) was added until pH 9-10. The resulting solids were removed by filtration through a Celite pad and the filtrate was concentrated in vacuo. The residue was redissolved in dichloromethane and the aqueous phase was separated. The organic phase was dried over MgSO₄, filtered, and concentrated in vacuo to yield benzyl 5-amino-1,2-dihydro-1′H-spiro[indole-3,4′-piperidine]-1′-carboxylate. MS (ES+): m/z 338 [M+1].

### Example 17

### N-(2′-Oxo-1′,2′-dihydrospiro[1,3-dioxolane-2,3′-indol]-5′-yl)-3-[(quinolin-4-ylmethyl)amino]thiophene-2-carboxamide

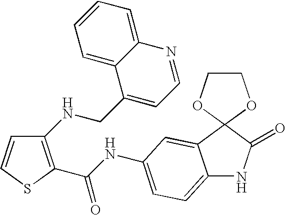

EXAMPLE 17 was prepared according to the procedure described for EXAMPLE 15 using 5′-aminospiro[1,3-dioxolane-2,3′-indol]-2′(1′H)-one (compound 28 in the procedure below) instead of 5-amino-1′-methylspiro[indole-3,4′-piperidin]-2(1H)-one. MS (ES+) 473 [M+1].

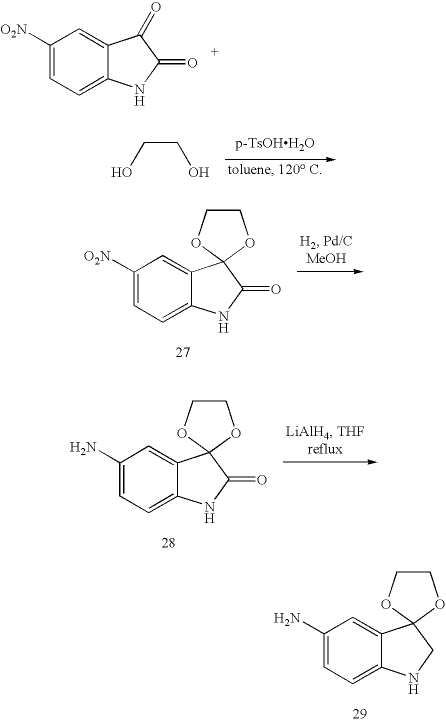

Part 1:

5′-Nitrospiro[1,3-dioxolane-2,3′-indol]-2′(1′H)-one (27): To a suspension of 5-nitro-1H-indole-2,3-dione (384 mg, 2.0 mmol) and ethylene glycol (248 mg, 4.0 mmol) in toluene was added p-TsOH.H₂O (19 mg, 0.1 mmol). The mixture was refluxed for 18 h with a Dean-Stark trap and the water produced was removed during the reaction. The reaction was then cooled to room temperature and the desired 5′-nitrospiro[1,3-dioxolane-2,3′-indol]-2′(1′H)-one precipitated out of solution and was collected by filtration. ¹H NMR (CDCl₃, 400 MHz): δ 4.37-4.41 (m, 2H), 4.56-4.60 (m, 2H), 6.95 (d, J=8.4 Hz, 1H), 7.70 (brs, 1H), 8.27 (d, J=2.0 Hz, 1H), 8.30 (dd, J=2.0, 8.4 Hz, 1H).

Part 2:

5′-Aminospiro[1,3-dioxolane-2,3′-indol]-2′(1′H)-one (28): To a suspension of 5′-nitrospiro[1,3-dioxolane-2,3′-indol]-2′(1′H)-one (379 mg, 1.6 mmol) was added 10% Pd/C (50 mg, 0.046 mmol). The solution was then deoxygenated and back filled with N₂ atmosphere (repeated 2×). The solution was then degassed and back filled with H₂ atmosphere (repeated 2×). The mixture was then stirred under H₂ atmosphere at rt overnight. The catalyst was removed by filtration through a Celite pad and the filtrate was concentrated in vacuo to yield 5′-aminospiro[1,3-dioxolane-2,3′-indol]-2′(1′H)-one. MS (ES+): m/z 207 [M+1].

Part 3:

1′,2′-Dihydrospiro[1,3-dioxolane-2,3′-indol]-5′-amine (29): To a suspension of LiAlH₄ (100 mg, 2.5 mmol) in THF (10 mL) was added 5′-aminospiro[1,3-dioxolane-2,3′-indol]-2′(1′H)-one (135 mg, 0.7 mmol). The mixture was stirred at rt for 30 min and at reflux for 3 h. After cooling to rt, water (1 mL) was added drop-wise to quench the reaction. The reaction was concentrated in vacuo and the residue was purified by silica gel chromatography (5% MeOH in CH₂Cl₂) to yield 1′,2′-dihydrospiro[1,3-dioxolane-2,3′-indol]-5′-amine. ¹H NMR (CD₃OD, 400 MHz): δ 3.55-3.58 (m, 2H), 3.88 (t, J=4.4 Hz, 2H), 4.04 (t, J=4.4 Hz, 2H), 6.69 (dd, J=2.0, 8.8 Hz, 1H), 6.75 (s, 1H), 6.07 (d, J=8.8 Hz, 1H). MS (ES+): m/z 193 [M+1].

### Example 18

### N-1′,2′-Dihydrospiro[1,3-dioxolane-2,3′-indol]-5′-yl-3-[(pyridin-4-ylmethyl)amino]thiophene-2-carboxamide

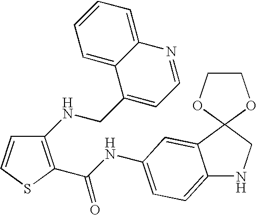

EXAMPLE 18 was prepared according to the procedure described for EXAMPLE 15 using 1′,2′-dihydrospiro[1,3-dioxolane-2,3′-indol]-5′-amine (compound 29 in the procedure above) instead of 5-amino-1′-methylspiro[indole-3,4′-piperidin]-2(1H)-one. MS (ES+) 459 [M+1].

### Example 19

### N-(2′-Oxo-1′,2′-dihydrospiro[1,3-dithiolane-2,3′-indol]-5′-yl)-3-[(quinolin-4-ylmethyl)amino]thiophene-2-carboxamide

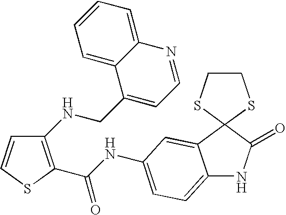

EXAMPLE 19 was prepared according to the procedure described for EXAMPLE 18 using ethylene thioglycol instead of ethylene glycol. MS (ES+) 505 [M+].

### Example 20

### N-(1,3-Dimethyl-2′-oxo-1′,2′-dihydrospiro[imidazolidine-2,3′-indol]-5′-yl)-3-[(quinolin-4-ylmethyl)amino]thiophene-2-carboxamide

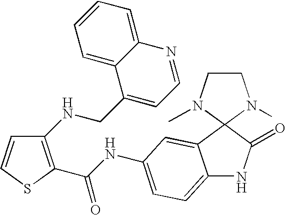

EXAMPLE 20 was prepared according to the procedure described for EXAMPLE 18 using N,N′-dimethylethylenediamine instead of ethylene glycol. MS (ES+) 499 [M+1].

### Example 21

### N-(2′-Oxo-1′,2′-dihydrospiro[1,3-dioxane-2,3′-indol]-5′-yl)-3-[(quinolin-4-ylmethyl)amino]thiophene-2-carboxamide

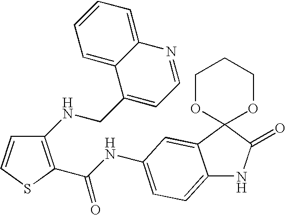

EXAMPLE 21 was prepared according to the procedure described for EXAMPLE 18 using propylene glycol instead of ethylene glycol. MS (ES+) 487 [M+1]

### Example 22

### 4-Methyl-N-(2′-oxo-1′,2′-dihydrospiro[1,3-dioxolane-2,3′-indol]-5′-yl)-3-[(quinolin-4-ylmethyl)amino]thiophene-2-carboxamide

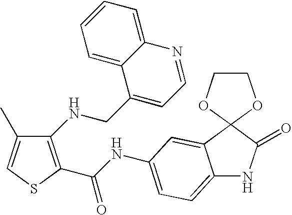

EXAMPLE 22 was prepared according to the procedure described above for EXAMPLE 17 using 3-amino-4-methyl-thiophene-2-carboxylic acid methyl ester instead of 3-amino-thiophene-2-carboxylic acid methyl ester. MS (ES+)487 [M+1].

### Example 23

### 4-Methyl-N-(2′-oxo-1′,2′-dihydrospiro[1,3-dithiolane-2,3′-indol]-5′-yl)-3-[(quinolin-4-ylmethyl)amino]thiophene-2-carboxamide

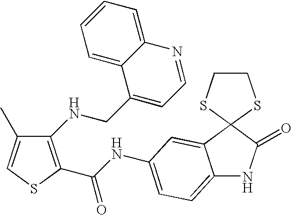

EXAMPLE 23 was prepared according to the procedure described above for EXAMPLE 19 using 3-amino-4-methyl-thiophene-2-carboxylic acid methyl ester instead of 3-amino-thiophene-2-carboxylic acid methyl ester. MS (ES+)519 [M+1].

### Example 24

### N-(1,3-Dimethyl-2′-oxo-1′,2′-dihydrospiro[imidazolidine-2,3′-indol]-5′-yl)-4-methyl-3-[(quinolin-4-ylmethyl)amino]thiophene-2-carboxamide

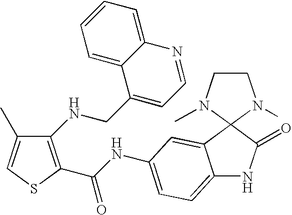

EXAMPLE 24 was prepared according to the procedure described above for EXAMPLE 20 using 3-amino-4-methyl-thiophene-2-carboxylic acid methyl ester instead of 3-amino-thiophene-2-carboxylic acid methyl ester. MS (ES+) 513 [M+1].

### Example 25

### 3-[(Quinolin-4-ylmethyl)amino]-N-(1,1′,3-trimethyl-2′-oxo-1′,2′-dihydrospiro[imidazolidine-2,3′-indol]-5′-yl)thiophene-2-carboxamide

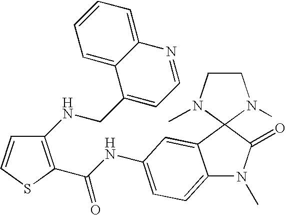

EXAMPLE 25 was prepared according to the procedure described for EXAMPLE 15 using 5′-amino-1,3-dimethylspiro[imidazolidine-2,3′-indol]-2′(1′H)-one (compound 31 in the procedure below) instead of 5-amino-1′-methylspiro[indole-3,4′-piperidin]-2(1H)-one. MS (ES+) 513 [M+1].

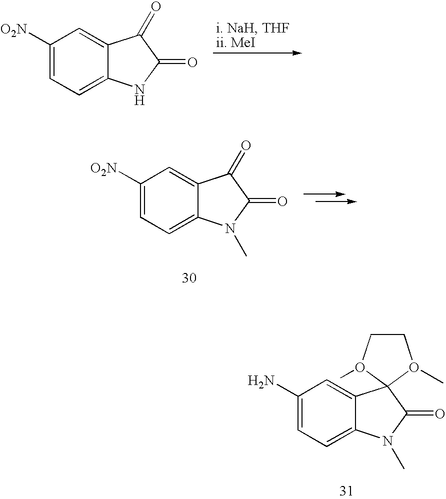

Part 1:

1-Methyl-5-nitro-1H-indole-2,3-dione (31): To a suspension of 5-nitro-1H-indole-2,3-dione (7.69 g, 40.0 mmol) in THF (100 mL) was added NaH (60% in mineral oil, 2.40 g, 60.0 mmol). After stirring for 1 h at rt, MeI (3.74 mL, 60.0 mmol) was added. The mixture was stirred at rt overnight, quenched with brine (80 mL), and extracted with dichloromethane (3×100 mL). The organic extracts were combined, dried over MgSO₄, filtered, and concentrated in vacuo. The crude solid was recrystallized from acetonitrile to yield methylated product 30. ¹H NMR (CDCl₃, 400 MHz): δ 3.07 (s, 3H), 6.59 (d, J=8.2 Hz, 1H), 6.67 (dd, J=2.4, 8.2 Hz, 1H), 6.74 (d, J=2.4 Hz, 1H). MS (ES+): m/z 207 [M+1]. This material was converted to 5′-amino-1,3-dimethylspiro[imidazolidine-2,3′-indol]-2′(1′H)-one (31) using the same procedure as in the synthesis of 1′,2′-dihydrospiro[1,3-dioxolane-2,3′-indol]-5′-amine (29).

### Example 26

### 4-Methyl-3-[(quinolin-4-ylmethyl)amino]-N-(1,1′,3-trimethyl-2′-oxo-1′,2′-dihydrospiro[imidazolidine-2,3′-indol]-5′-yl)thiophene-2-carboxamide

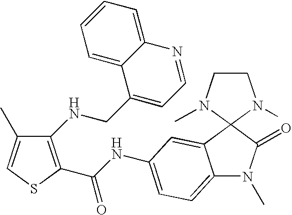

EXAMPLE 26 was prepared according to the procedure described above for EXAMPLE 25 using 3-amino-4-methyl-thiophene-2-carboxylic acid methyl ester instead of 3-amino-thiophene-2-carboxylic acid methyl ester. MS (ES+) 527 [M+1].

What is claimed is:

1. A compound represented by Formula (I): 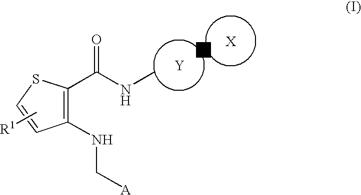
or a pharmaceutically acceptable salt or N-oxide thereof, wherein:
Y is heteroaryl or cycloC₃₋₁₀alkyl, either of which is optionally substituted by 1-5 independent R² substituents;
X is heteroaryl or heterocylyl, either of which is optionally substituted by 1-5 independent R²¹ substituents;
A is aryl, heteroaryl, cycloC₃₋₁₀alkyl, heterocyclyl, cycloC₃₋₁₀alkenyl, or heterocycloalkenyl, each of which is optionally substituted by 1-5 independent R³ substituents;
R¹ is C₀₋₆alkyl, halogen, or haloalkyl;
R², R²¹, and R³ each independently is C₀₋₆alkyl, cycloC₃₋₁₀alkyl, oxo, halogen, haloalkyl; cyanoC₀₋₆alkyl, nitroC₀₋₆alkyl, hydroxyC₀₋₆allyl, —C₀₋₆alkyl-N(C₀₋₆alkyl)(C₀₋₆alkyl), —N(C₀₋₆alkyl)-N(C₀₋₆alkyl)(C₀₋₆alkyl), —N(C₀₋₆alkyl)-N(C₀₋₆alkyl)(acyl), acylC₀₋₆alkyl, substituted acyl, guanidinoC₀₋₆alkyl, hydroxyiminoC₀₋₆alkyl, acylaminoC₀₋₆alkyl, substituted acylamino, acyloxyC₀₋₆alkyl, substituted acyloxy, arC₀₋₆alkyl, substituted arC₀₋₆alkyl, heteroarylC₀₋₆alkyl, substituted heteroarylC₀₋₆alkyl, heterocyclylC₀₋₆alkyl, cyanoaminoC₀₋₆alkyl, C₀₋₆alkylhydrazino, heterocyclylamino, arC₀₋₆alkylhydrazino, alkylsulfonylC₀₋₆alkyl, arC₀₋₆alkylsulfonylC₀₋₆alkyl, alkylsulfinylC₀₋₆alkyl, alkylsulfonamidoC₀₋₆alkyl, arC₀₋₆alkylsulfonamidoC₀₋₆alkyl, aminoC₀₋₆alkylsulfonyl, C₀₋₆alkylaminosulfonyl, acylC₁₋₆alkylsulfonyl, heterocyclylsulfonyl, aminoC₀₋₆alkylsulfinyl, acylC₁₋₆alkylsulfinyl, silyl, siloxy, alkenoxy, alkynoxy, C₂₋₆alkenyl, acylC₂₋₆alkenyl, C₂₋₆alkynyl, acylC₂₋₆alkynyl, hydroxyC₂₋₆alkynyl, aminoC₂₋₆alkynyl, C₁₋₆alkoxyC₀₋₆alkyl, C₁₋₆alkylthioC₀₋₆alkyl, hydroxyC₁₋₆alkoxyC₀₋₆alkyl, hydroxyC₁₋₆alkylthioC₀₋₆alkyl, acylC₁₋₆alkoxyC₀₋₆alkyl, acylC₁₋₆alkylthioC₀₋₆alkyl, C₀₋₆alkylaminoC₁₋₆alkoxyC₀₋₆alkyl, C₀₋₆alkylaminoC₁₋₆alkylthioC₀₋₆alkyl, acylaminoC₁₋₆alkoxyC₀₋₆alkyl, acylaminoC₁₋₆alkylthioC₀₋₆alkyl, arC₀₋₆alkylaminoC₀₋₆alkyl, arC₀₋₆alkylthioC₀₋₆alkyl, arC₀₋₆alkoxyC₀₋₆alkyl, arC₀₋₆alkylamino, arC₀₋₆alkylaminoC₀₋₆alkyl, arC₀₋₆alkylthio, substituted aro-alkoxy, substituted arC₀₋₆alkylthio, or substituted arC₀₋₆alkoxy; and
provided that the compound is not
cis-N-(4-methyl-5-oxo-1,4-diazaspiro[S 0.5]undec-9-yl)-3-[(quinolin-4-ylmethyl)amino]thiophene-2-carboxamide,
cis-N-(2-benzyl-3-oxo-1,2-diazaspiro[4.5]dec-8-yl)-3-[(quinolin-4-ylmethyl)amino]thiophene-2-carboxamide,
N-(3-benzyl-2,4-dioxo-1,3-diazaspiro[4.5]dec-8-yl)-3-[(quinolin-4-ylmethyl)amino]thiophene-2-carboxamide,
cis-N-(2,4-dioxo-1,3-diazaspiro[4.5]dec-8-yl)-3-[(quinolin-4-ylmethyl)amino]thiophene-2-carboxamide,
N-[3-(3-methylbutyl)-2,4-dioxo-1,3-diazaspiro[4.5]dec-8-yl]4-methyl-3-[(quinolin-4-ylmethyl)amino]thiophene-2-carboxamide, or
benzyl 5-{[(3-[(quinolin-4-ylmethyl)amino]thien-2-yl)carbonyl]amino}-2-oxo-1,2-dihydro-1′H-spiro[indole-3,4′-piperidine]-1′-carboxylate.

2. The compound according to claim 1 wherein Y is cycloC₃₋₁₀alkyl optionally substituted by 1-5 independent R² substituents; and X is heterocyclyl or heteroaryl, optionally substituted by 1-5 independent R²¹ substituents.

3. The compound according to claim 2 wherein Y is cyclohexyl optionally substituted by 1-5 independent R² substituents.

4. The compound according to claim 3 wherein A is heteroaryl.

5. The compound according to claim 4 wherein A is

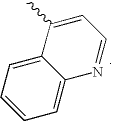

6. The compound according to claim 5 wherein R¹ is C₀₋₆alkyl.

7. The compound according to claim 1 wherein Y is heteroaryl optionally substituted by 1-5 independent R² substituents; and X is heterocyclyl optionally substituted by 1-5 independent R²¹ substituents.

8. The compound according to claim 1 wherein Y is cycloC₃₋₁₀alkyl optionally substituted by 1-5 independent R² substituents; and X is heterocyclyl optionally substituted by 1-5 independent R²¹ substituents.

9. The compound according to claim 7 wherein A is heteroaryl.

10. The compound according to claim 9 wherein A is

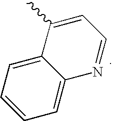

11. The compound according to claim 10 wherein R¹ is C₀₋₆alkyl.

12. A composition comprising
a compound according to claim 1, or a pharmaceutically acceptable salt or N-oxide thereof; and
a pharmaceutically acceptable carrier.

13. A composition comprising
a compound according to claim 1, or a pharmaceutically acceptable salt or N-oxide thereof; and
an anti-neoplastic, anti-tumor, anti-angiogenic, or chemotherapeutic agent.

14. A composition comprising
a compound according to claim 1, or a pharmaceutically acceptable salt or N-oxide thereof; and
a cytotoxic cancer therapeutic agent.

15. A composition comprising
a compound according to claim 1, or a pharmaceutically acceptable salt or N-oxide thereof; and
an angiogenesis inhibiting cancer therapeutic agent.

16. A compound consisting of
N-1,4-dioxaspiro[4.5]dec-8-yl-3-[(quinolin-4-ylmethyl)amino]thiophene-2-carboxamide;
cis-N-1-oxa-4-thiaspiro[4.5]dec-8-yl-3-[(quinolin-4-ylmethyl)amino]thiophene-2-carboxamide;
trans-N−1-oxa-4-thiaspiro[4.5]dec-8-yl-3-[(quinolin-4-ylmethyl)amino]thiophene-2-carboxamide;
N-(2-methyl-3-oxo-1,2-diazaspiro[4.5]dec-8-yl)-3-[(quinolin-4-ylmethyl)amino]thiophene-2-carboxamide;
trans-N-(2-benzyl-3-oxo-1,2-diazaspiro[4.5]dec-8-yl)-3-[(quinolin-4-ylmethyl)amino]thiophene-2-carboxamide;
cis-N-(1,4-dimethyl-5-oxo-1,4-diazaspiro[5.5]undec-9-yl)-3-[(quinolin-4-ylmethyl)amino]thiophene-2-carboxamide;
trans-N-(1,4-dimethyl-5-oxo-1,4-diazaspiro[5.5]undec-9-yl)-3-[(quinolin-4-ylmethyl)amino]thiophene-2-carboxamide;
cis-N-(3′-oxo-3′,4′-dihydro-1′H-spiro[cyclohexane-1,2′-quinoxalin]4-yl)-3-[(quinolin-4-ylmethyl)amino]thiophene-2-carboxamide;
trans-N-(3′-oxo-3′,4′-dihydro-1′H-spiro[cyclohexane-1,2′-quinoxalin]4-yl)-3-[(quinolin-4-ylmethyl)amino]thiophene-2-carboxamide;
N-(3-oxo-2-azaspiro[4.5]dec-8-yl)-3-[(quinolin-4-ylmethyl)amino]thiophene-2-carboxamide;
N-1,4-dioxaspiro[4.5]dec-8-yl-4-methyl-3-[(quinolin-4-ylmethyl)amino]thiophene-2-carboxamide;
trans-N-(2,4-dioxo-1,3-diazaspiro[4.5]dec-8-yl)-3-[(quinolin-4-ylmethyl)amino]thiophene-2-carboxamide;
N-(3-methyl-2,4-dioxo-1,3-diazaspiro[4.5]dec-8-yl)-3-[(quinolin-4-ylmethyl)amino]thiophene-2-carboxamide;
N-[3-(3-methylbutyl)-2,4-dioxo-1,3-diazaspiro[4.5]dec-8-yl]-3-[(quinolin-4-ylmethyl)amino]thiophene-2-carboxamide;
N-(1′-methyl-2-oxo-1,2-dihydrospiro[indole-3,4′-piperidin]-5-yl)-3-[(quinolin-4-ylmethyl)amino]thiophene-2-carboxamide;
N-(2′-oxo-1′,2′-dihydrospiro[1,3-dioxolane-2,3′-indol]-5′-yl)-3-[(quinolin-4-ylmethyl)amino]thiophene-2-carboxamide;
N-1′,2′-dihydrospiro[1,3-dioxolane-2,3′-indol]-5′-yl-3-[(pyridin-4-ylmethyl)amino]thiophene-2-carboxamide;
N-(2-oxo-1,2,2′,3′,5′,6′-hexahydrospiro[indole-3,4′-pyran]-5-yl)-3-[(quinolin-4-ylmethyl)amino]thiophene-2-carboxamide;
N-(2′-oxo-1′,2′-dihydrospiro[1,3-dithiolane-2,3′-indol]-5′-yl)-3-[(quinolin-4 ylmethyl)amino]thiophene-2-carboxamide;
N-(1,3-dimethyl-2′-oxo-1′,2′-dihydrospiro[imidazolidine-2,3′-indol]-5′-yl)-3-[(quinolin-4-ylmethyl)amino]thiophene-2-carboxamide;
3-[(quinolin-4-ylmethyl)amino]-N-(1,1′,3-trimethyl-2′-oxo-1′,2′-dihydrospiro[imidazolidine-2,3′-indol]-5′-yl)thiophene-2-carboxamide;
N-(2′-oxo-1′,2′-dihydrospiro[1,3-dioxane-2,3′-indol]-5′-yl)-3-[(quinolin-4-ylmethyl)amino]thiophene-2-carboxamide;
4-methyl-N-(2′-oxo-1′,2′-dihydrospiro[1,3-dioxolane-2,3′-indol]-5′-yl)-3-[(quinolin-4-ylmethyl)amino]thiophene-2-carboxamide;
4-methyl-N-(2′-oxo-1′,2′-dihydrospiro[1,3-dithiolane-2,3′-indol]-5′-yl)-3-[(quinolin-4-ylmethyl)amino]thiophene-2-carboxamide;
N-(1,3-dimethyl-2′-oxo-1′,2′-dihydrospiro[imidazolidine-2,3′-indol]-5′-yl)-4-methyl-3-[(quinolin-4-ylmethyl)amino]thiophene-2-carboxamide;
4-methyl-3-[(quinolin-4-ylmethyl)amino]-N-(1,1′,3-trimethyl-2′-oxo-1′,2′-dihydrospiro[imidazolidine-2,3′-indol]-5′-yl)thiophene-2-carboxamide; or
a pharmaceutically acceptable salt, or N-oxide, thereof.

17. A compound consisting of 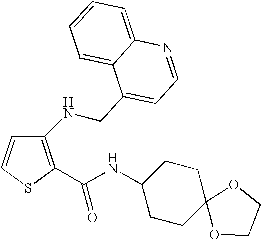 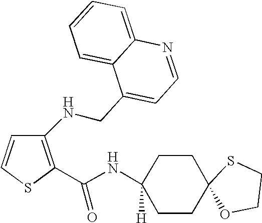 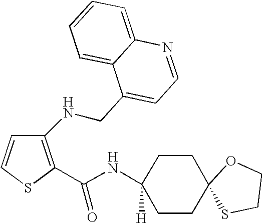 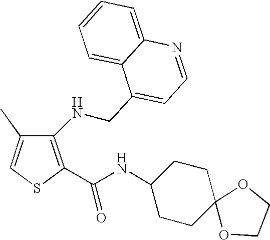 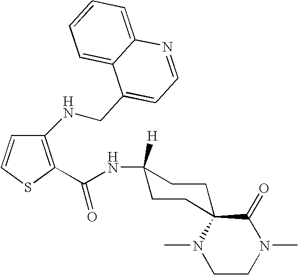 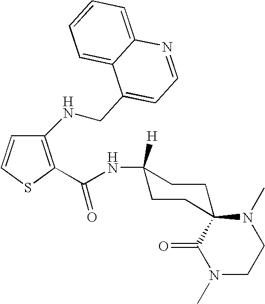 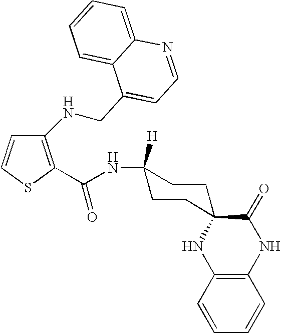 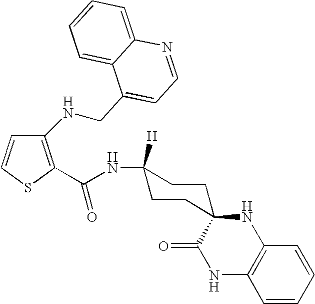 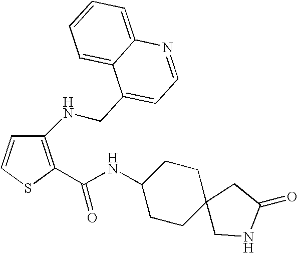 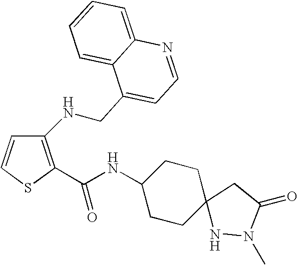 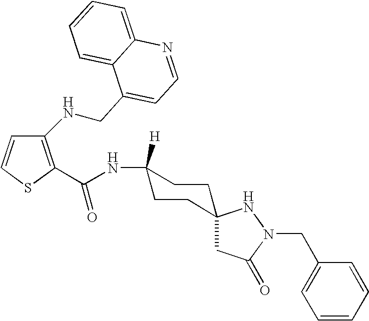 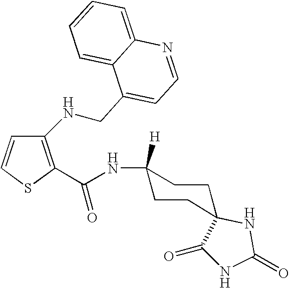 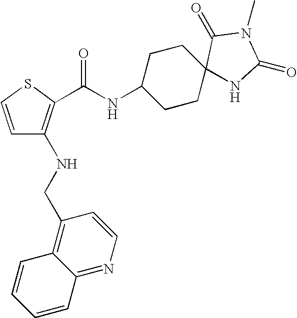 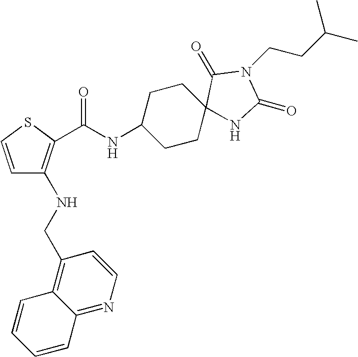
or a pharmaceutically acceptable salt, or N-oxide, thereof.

18. A compound consisting of 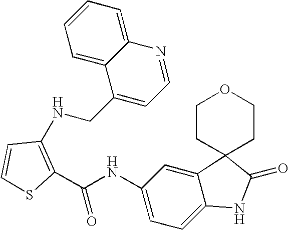 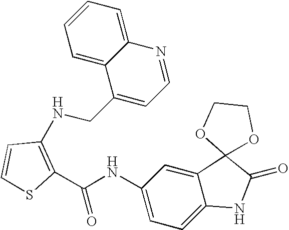 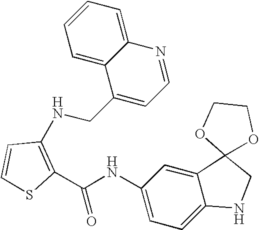 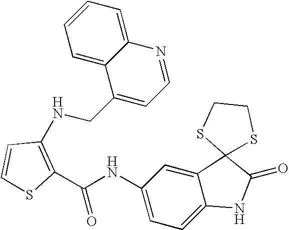 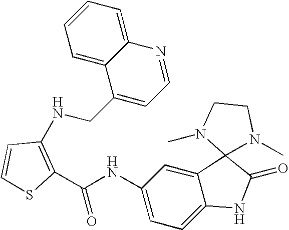 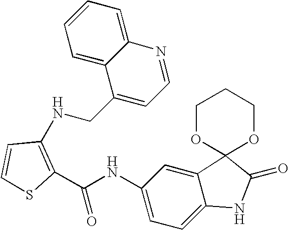 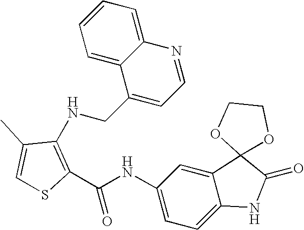 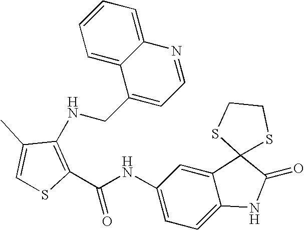 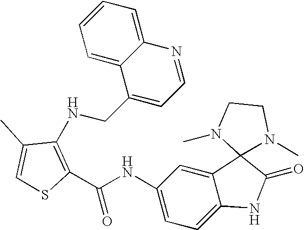 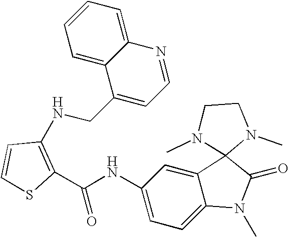 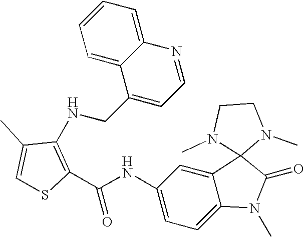 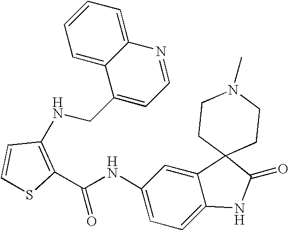
or a pharmaceutically acceptable salt, or N-oxide, thereof.

19. A method of treatment of hyperproliferative disorder comprising a step of administering an effective amount of the compound according to claim 1.

20. The method of claim 19, further comprising the step of administering an anti-neoplastic, anti-tumor, anti-angiogenic, or chemotherapeutic agent.

21. The method of claim 19 wherein the hyperproliferative disorder is breast cancer, head cancer, or neck cancer.

22. The method of claim 19 wherein the hyperproliferative disorder is gastrointestinal cancer.

23. The method of claim 19 wherein the hyperproliferative disorder is leukemia.

24. The method of claim 19 wherein the hyperproliferative disorder is ovarian, bronchial, lung, or pancreatic cancer.

25. The method of claim 19 wherein the hyperproliferative disorder is mastocytosis/mast cell leukemia, gastrointestinal stromal tumors (GIST), germ cell tumors, small cell lung carcinoma (SCLC), sinonasal natural killer/T-cell lymphoma, testicular cancer (seminoma), thyroid carcinoma, malignant melanoma, ovarian carcinoma, adenoid cystic carcinoma, acute myelogenous leukemia (AML), breast carcinoma, pediatric T-cell acute lymphoblastic leukemia, neuroblastoma, mast cell leukemia, angiosarcoma, anaplastic large cell lymphoma, endometrial carcinoma, and prostate carcinoma.
